(19) United States
(12) Patent Application Publication
(10) Pub. No.: US 2021/0147545 A1
(43) Pub. Date: May 20, 2021

(54) IMMUNOTOXINS FOR USE IN TREATING CANCER

(71) Applicant: OSLO UNIVERSITETSSYKEHUS HF, Oslo (NO)

(72) Inventors: Yvonne ANDERSSON, Nesodden (NO); Øystein FODSTAD, Oslo (NO); Svein DUELAND, Oslo (NO); Kjersti FLATMARK, Blommenholm (NO)

(21) Appl. No.: 16/633,824

(22) PCT Filed: Jul. 24, 2018
(86) PCT No.: PCT/EP2018/070072

§ 371 Date: Jan. 24, 2020

(30) Foreign Application Priority Data
Jul. 24, 2017 (EP) 17182878.3

(51) Int. Cl. C07K 16/28; A61P 35/00; A61K 47/68

(52) U.S. Cl. CPC C07K 16/2827; A61K 47/6829; C07K 16/2818; A61P 35/00

(57) ABSTRACT

The present invention relates generally to immunotoxins. More particularly, the invention relates to immunotoxins (e.g. immunotoxins that comprise an antibody that binds to EpCAM or binds to MUC-1) for use in the treatment of cancer. The invention also relates to a method for treating cancer, said method comprising administering to a subject in need thereof a therapeutically effective amount of an immunotoxin. The invention also relates to the use an immunotoxin in the manufacture of a medicament for treating cancer.

The present invention relates generally to immunotoxins. More particularly, the invention relates to immunotoxins for use in the treatment of cancer.

Cancer treatment is still one of the biggest unmet medical needs. While there have been advances in cancer therapy during the last decades, cancer remains a leading cause of death. Thus, the demand for new cancer therapies is ever increasing.

One of the hallmarks of cancer is to try to escape an anti-tumor immune response. Lately, various approaches to overcome immune resistance have been applied clinically in several tumor types. However, in spite of some cases with unforeseen durable responses, only about 25% of all patients respond to immuno-oncology drugs. To improve response rates and overcome resistance new drugs and combinations are being evaluated in the clinic, and compounds with different mechanisms of immune-stimulation are investigated.

What are needed in the art are new cancer therapies which are able to make use of the immune system of the subject being treated in order for there to be, or to enhance, a clinically beneficial anti-cancer immune response.

The present inventors have surprisingly found that immunotoxins can be useful for this purpose. Immunotoxins typically have an antibody component conjugated to a cytotoxic biological molecule.

Classically, immunotoxins exert an anti-cancer effect by binding to a target cancer cell, internalization of the toxin and the induction of cell death by the cytotoxic molecule via the catalytic inactivation of vital processes, such as protein synthesis, and by directly inducing apoptosis. However, it is thought that immunotoxin cancer treatments can be hampered by the inaccesibility of some cancer cells (for example in solid tumours) meaning that the immunotoxins are unable to exert their effect on all cells in a cancer. It is also thought that immunotoxins can be hampered by a host immune response to the immunotoxins themselves.

However, surprisingly, the present inventors have found that immunotoxins can induce immunogenic cell death (ICD) in cancer cells and accordingly can elicit a therapeutically beneficial anti-cancer immune response. More specifically, the treatments can provide a significant improvement in survival prospects (life expectancy) for cancer patients.

Thus, in one aspect, the present invention provides an immunotoxin for use in treating cancer, wherein the immunotoxin induces immunogenic cell death of cancer cells in a subject.

Immunotoxins comprise (or consist of) an antibody (or antibody moiety or antibody component) conjugated to (or coupled to) a toxic biological molecule (a cytotoxic biological molecule). Typically, and preferably, the toxic biological molecule is a protein or peptide. The antibody may be conjugated to a toxic biological molecule by any appropriate means.

The preparation and structure of immunotoxins is generally well known in the art. Immunotoxins that have been produced (or generated) by any appropriate means (e.g. as described elsewhere herein) may be used in accordance with the invention.

For example, in the preparation of immunotoxins recombinant expression may be employed (and the resulting immunotoxin is a recombinant immunotoxin). In recombinant methods, nucleic acid sequences encoding the chosen antibody (or part thereof) and toxic biological molecule may be attached in-frame in an expression vector. Recombinant expression thus results in translation of the nucleic acid to yield the desired immunotoxin as a fusion protein. In some embodiments of the present invention, the immunotoxin is not a recombinant immunotoxin, i.e. it is not a fusion protein (the antibody component and the toxic moiety may preferably not be linked together via a peptide bond in the same single protein molecule).

In other examples, chemical cross-linkers or avidin:biotin bridges may join the toxic biological molecule to the chosen antibody.

In some preferred embodiments, the antibody is attached to (or conjugated to or linked to) the toxic biological molecule by (or via) a covalent bond. In particularly preferred embodiments the antibody moiety is attached to the toxic biological molecule by (or via) a thioether bond. Such a bond may, for example, be formed with (or using) the reagent sulfo-SMCC (sulfo-succinimidyl-4-(N-maleimidomethyl) cyclohexane-1-carboxylate) or the reagent SMCC (succinimidyl-4-(N-maleimidomethyl) cyclohexane-1-carboxylate). Such a bond (linkage) is described, for example, by Godal et al. (Int. J. Cancer, 1992, 52:631-635). Thus in some embodiments, conjugation of the antibody to the toxic biological molecule (typically a protein or peptide) is done using a sulfo-SMCC method or a SMCC method. Such methods of conjugation are well-known in the art.

In some preferred embodiments, the antibody molecule and the toxic biological molecule are produced separately and then subsequently conjugated together (e.g. by (or via) a thioether bond, e.g. as described elsewhere herein). In some preferred embodiments, the antibody molecule and the toxic biological molecule are conjugated (attached together) via a linker (or cross-linker). Thus, in preferred embodiments, immunotoxins used in accordance with the invention comprise (or consist of) an antibody molecule, a linker (or cross-linker) and a toxic biological molecule (with the linker acting to conjugate or attach the antibody molecule to the toxic biological molecule). Suitable linkers are known in the art and any appropriate linker may be employed.

In some embodiments, the linker (or linkage or attachment or conjugation) has 5-50 carbon atoms, more preferably 10-25 carbon atoms, e.g. 12-16 carbon atoms (e.g. 12 carbon atoms). In some embodiments, the linker (or linkage or attachment or conjugation) in the immunotoxin comprises one or more cyclic groups (e.g. one or two cyclic groups, preferably two cyclic groups). In some embodiments, the linker (or linkage or attachment or conjugation) in the immunotoxin comprises a maleimide group. In some embodiments, the linker (or linkage or attachment or conjugation) between the antibody molecule and the toxic biological molecule comprises a sulphur atom, preferably a sulphur atom in a thioether bond. In some embodiments, the linker (or linkage or attachment or conjugation) comprises a chemical group (or moiety) obtained by treating the antibody molecule or toxic biological molecule (as the case may be) with the reagent SMCC or the reagent sulfo-SMCC.

In some embodiments, the conjugation (or linkage or linker or attachment) between the antibody molecule and the toxic biological molecule is a conjugation produced (or obtained or formed) by contacting (or reacting) an antibody that has been treated (or derivatised or reduced) using (or with) a reducing agent (e.g. DTT, dithiothreitol) to generate one or more free sulfhydryl (SH) groups on the antibody molecule with a toxic biological molecule that has been treated (or derivatised) using (or with) a reagent that comprises a maleimide group (e.g. sulfo-SMCC (sulfo-succinimidyl-4-(N-maleimidomethyl) cyclohexane-1-carboxylate) or SMCC (succinimidyl-4-(N-maleimidomethyl) cyclohexane-1-carboxylate)). In some embodiments, the conjugation (or linkage or linker or attachment) between the antibody molecule and the toxic biological molecule is a conjugation produced (or obtained) by contacting (or reacting) an antibody that has been treated (or derivatised or reduced) using (or with) DTT (dithiothreitol) to generate one or more free sulfhydryl (SH) groups on the antibody molecule with a toxic biological molecule that has been treated (or derivatised) using (or with) the SMCC (succinimidyl-4-(N-maleimidomethyl) cyclohexane-1-carboxylate) reagent. In such embodiments the antibody molecule and the toxic biological molecule(s) are conjugated via a thioether bond(s) (e.g. via the sulphur atom of a sulfhydryl group). A thioether bond is typically formed between the sulfhydryl group and the maleimide group. Such a thioether bond(s) may be formed on the free sulfhydryl group(s) after a hinge disulphide bond(s) of the antibody has been reduced. Thus, a toxic biological molecule may be attached to an antibody molecule at the position of a disulphide bond(s) in the hinge region of the antibody.

In some embodiments, the conjugation (or linkage or linker or attachment) between the antibody molecule and the toxic biological molecule is a conjugation produced (or obtained or formed) by contacting (or reacting) a toxic biological molecule that has been treated (or derivatised or reduced)) using (or with) a reducing agent (e.g. DTT, dithiothreitol) to generate one or more free sulfhydryl (SH) groups on the toxic biological molecule with an antibody that has been treated (or derivatised) using (or with) a reagent that comprises a maleimide group (e.g. sulfo-SMCC (sulfo-succinimidyl-4-(N-maleimidomethyl) cyclohexane-1-carboxylate) or SMCC (succinimidyl-4-(N-maleimidomethyl) cyclohexane-1-carboxylate)). In some embodiments, the conjugation (or linkage or linker or attachment) between the antibody molecule and the toxic biological molecule is a conjugation produced (or obtained) by contacting (or reacting) a toxic biological molecule that has been treated (or derivatised or reduced) using (or with) DTT (dithiothreitol) to generate one or more free sulfhydryl (SH) groups on the toxic biological molecule with an antibody molecule that has been treated (or derivatised) using (or with) the SMCC (succinimidyl-4-(N-maleimidomethyl) cyclohexane-1-carboxylate) reagent. In some such embodiments the antibody molecule and the toxic biological molecule are conjugated via a thioether bond (e.g. via the sulphur atom of a sulfhydryl group). A thioether bond is typically formed between the sulfhydryl group and the maleimide group.

Appropriate methods and reaction conditions for conjugating antibodies to other biological molecules are well-known in the art and any suitable method may be used. For example, if a SMCC or a sulfo-SMCC method is used (e.g. as described above), the conjugation reaction (or contacting) may be carried out at pH 6.5-7.5.

Thus, in some preferred embodiments the antibody molecule and the toxic biological molecule are not linked together by a peptide bond.

Immunotoxins are not to be considered synonymous with antibody-drug conjugates (ADCs). As mentioned above, immunotoxins generally comprise (or consist of) an antibody conjugated to a toxic protein- or peptide-based molecule (cytotoxic biological molecule). In contrast, ADCs generally comprise an antibody conjugated to a toxic chemical entity (non-biological toxic chemical entity or drug, e.g. the non-biological toxic chemical entity is not a protein or peptide). ADCs may also be defined as monoclonal antibodies linked to small molecule drugs, that can target a specific cancer cell and that have reduced systemic toxicity.

In accordance with this aspect of the present invention, the immunotoxin induces immunogenic cell death (ICD) of cancer cells in a subject.

Immunogenic cell death is a form of cell death caused by some agents. Immunogenic cell death is not the same as, i.e. is distinct from, apoptosis (which is typically non-immunogenic, e.g. caspase/PARP mediated apoptosis) or inhibition of protein synthesis. Immunogenic cell death of cancer cells can induce an effective anti-tumour immune response, e.g. through activation (or maturation) of dendritic cells (DCs) and consequent activation of a T cell response.

Classically with immunotoxin cancer treatments, the antibody component targets the immunotoxin to cancer cells expressing the antigen to which the antibody component is directed, and when internalised, the toxin effector moiety triggers cell death by catalytic inactivation of vital processes, such as protein synthesis, and by directly inducing apoptosis. “Classical” mechanisms of action thus typically involve the inhibition of protein synthesis and the induction of apoptosis. Without wishing to be bound by theory, the use of an immunotoxin that induces immunogenic cell death of cancer cells in a subject is particularly useful because the immunotoxin does not need to penetrate an entire cancer (or tumour) and exert its cytotoxic effect directly on all cells in the cancer or tumour (via classical mechanisms), but rather the immunotoxin can target a limited number of cancer cells and induce immunogenic cell death and the subject's immune response can be relied upon to target the remainder of the tumour. Thus, alternatively viewed, immunotoxin therapies in accordance with the present invention can have an indirect effect on cancer, by indirectly targeting cancer cells via the stimulation (or enhancement) of a subject's immune response (e.g. T-cell or NK cell response). Thus, immunotoxins can exert an indirect anti-cancer effect rather than solely a classical direct effect. Immunotoxin induced immunogenic cell death of cancer cells can be considered an indirect effect as it involves the raising of an immune response (e.g. it involves dendritic cells and T cells). Conversely, classical (i.e. non-ICD) immunotoxin mediated cancer cell killing involves only the immunotoxin, there is no anti-cancer immune response component.

Immunogenic cell death in accordance with the present invention may typically be characterised by the activation (or maturation) of dendritic cells (DCs) in a subject being treated with the immunotoxin and typically by the activation of a T cell response (for example in the tumour microenvironment).

Typically, immunotoxin administration in accordance with the present invention may induce a Th1 cytokine response (e.g. a significant Th1 cytokine response).

Immunogenic cell death in accordance with the present invention may be characterised by the presence of one or more pro-inflammatory cytokines, particularly one or more (or all) of the Th1-type pro-inflammatory cytokines selected from the group consisting of IL-2 (interleukin-2), IL-6 (interleukin-6), IL-12 (interleukin-12), IFNγ (interferon-γ) and TNFα (tumour necrosis factor α), and/or one or both of the Th1 related cytokines IL-1β (interleukin-1β) or GM-CSF (granulocyte-macrophage colony-stimulating factor).

Immunogenic cell death in accordance with the present invention may be characterised by an increase in the level of one or more (or all) of the cytokines selected from the group consisting of IL-2, IL-12, IFNγ, TNFα, IL-1β and GM-CSF.

Immunogenic cell death in accordance with the present invention may be characterised by an increase in the level of one or both of the cytokines IL-2 or TNFα.

Immunogenic cell death in accordance with the present invention may also be characterised by an increase in the level of one or more (or all) of the cytokines selected from the group consisting of IL-2, IL-12, and GM-CSF (for example when the immunotoxin is used as the sole active agent in the treatment regimen).

The “increase” in cytokine levels mentioned above may be any measurable increase or elevation of the level of the cytokine in question, e.g. when the cytokine in question is compared with a control level. Preferably, the level is significantly increased, compared to the level found in an appropriate control sample or subject. More preferably, the significantly increased levels are statistically significant, preferably with a probability value of <0.05 or <0.01. Suitable control levels (control samples or subjects) are described in the Example section. An example of a control level is the level of the cytokine in question pre-treatment (prior to first administration of the immunotoxin). Thus, a control level may be the level of the cytokine in question in the same subject pre-treatment (prior to first administration of the immunotoxin). Appropriate control levels (or control samples or values) could be readily chosen by a person skilled in the art. Appropriate control “values” could also be readily determined without running a control “sample” in every test, e.g. by reference to the range for normal subjects or “pre-treatment” subjects known in the art.

The level of one or more cytokines may be determined at any appropriate time, for example about one to seven days (e.g. about 1 day, about 2 days or about 4 days), about one week, about two weeks, about three weeks, about four weeks, about six weeks or about eight weeks (preferably two weeks) after the first administration of the immunotoxin.

The presence and/or level of one or more cytokines may be determined by any appropriate means, in any appropriate sample type. An appropriate and preferred sample type in which the presence and/or level of one or more cytokines may be determined is serum. Samples (e.g. serum) may be frozen prior to being processed. An appropriate and preferred technique for determining the presence and/or level of one or more cytokines is ELISA, for example as described in the Examples herein.

As mentioned above, immunogenic cell death may be characterised by the activation (or maturation) of dendritic cells. This may be determined by any appropriate means, in any appropriate sample type, and a person skilled in the art is familiar with suitable techniques (e.g. flow cytometry e.g. with cells stained via a fluorescently labelled CD9 antibody and a fluorescently labelled CD14 antibody, or e.g. with a fluorescently labelled CD14 antibody, or e.g. via a fluorescently labelled CD86 antibody, or e.g. via a fluorescently labelled CD9 antibody and a fluorescently labelled CD86 antibody, or e.g. via a fluorescently labelled CD9 antibody and a fluorescently labelled CD14 antibody and a fluorescently labelled CD86 antibody, for example in accordance with the Example section herein).

For example, a decrease (e.g. a significant decrease) in the level of the marker CD14 on dendritic cells is indicative that dendritic cells are mature or activated (or maturing or being activated). The marker CD14 is a marker of immature dendritic cells. A decrease in the level of the marker CD14 on dendritic cells may be as assessed in comparison with any appropriate control level or reference value (e.g. in comparison with the level of CD14 on dendritic cells in the same subject, or a sample from the same subject, prior to immunotoxin treatment). Other appropriate control levels (or control samples) could be readily chosen by a person skilled in the art, for example, an appropriate control could be the level of CD14 on DCs in a subject (e.g. in a sample from a subject) that had not been treated with the immunotoxin. Appropriate control “values” could also be readily determined without running a control “sample” in every test, e.g. by reference to the range for normal subjects known in the art.

A “decrease” in CD14 levels mentioned above may be any measurable decrease or reduction of the level of CD14, e.g. as compared with a control level or value. Preferably, the level is significantly decreased, compared to the level found in an appropriate control sample or subject. More preferably, the significantly decreased levels are statistically significant, preferably with a probability value of <0.05 or <0.01 or <0.0025 or <0.0010 or 0.0001.

By way of another example, an increase (e.g. a significant increase) in the level of the marker CD86 on dendritic cells is indicative that dendritic cells are mature or activated (or maturing or being activated). The marker CD86 is a marker of mature dendritic cells. An increase in the level of the marker CD86 on dendritic cells may be as assessed in comparison with any appropriate control level or reference value (e.g. in comparison with the level of CD86 on dendritic cells in the same subject, or a sample from the same subject, prior to immunotoxin treatment). Other appropriate control levels (or control samples) could be readily chosen by a person skilled in the art, for example, an appropriate control could be the level of CD86 on DCs in a subject (e.g. in a sample from a subject) that had not been treated with the immunotoxin. Appropriate control “values” could also be readily determined without running a control “sample” in every test, e.g. by reference to the range for normal subjects known in the art.

An “increase” in CD86 levels mentioned above may be any measurable increase or elevation of the level of CD86, e.g. as compared with a control level or value. Preferably, the level is significantly increased, compared to the level found in an appropriate control sample or subject. More preferably, the significantly increased levels are statistically significant, preferably with a probability value of <0.05 or <0.01 or <0.0025 or <0.0010 or 0.0001.

As mentioned elsewhere herein, immunogenic cell death in accordance with the present invention may be characterised by the activation (or promotion or enhancement or increase or induction) of a T cell response (for example in the tumour microenvironment). In some embodiments, the T cell response is characterised by an increase in the CD8⁺ T cell population (or killer T cell (CD8⁺) population or activated CD8⁺ killer T cell population), e.g. in a tumour microenvironment.

An increase in the CD8⁺ T cell population may be as assessed in comparison with any appropriate control level or reference value (e.g. in comparison with the CD8⁺ T cell population in the same subject, or a sample from the same subject, prior to immunotoxin treatment). Other appropriate control levels (or control samples) could be readily chosen by a person skilled in the art, for example, an appropriate control could be CD8⁺ T cell population size in a subject (e.g. in a sample from a subject) that had not been treated with the immunotoxin. Appropriate control “values” could also be readily determined without running a control “sample” in every test, e.g. by reference to the range for normal subjects known in the art.

An “increase” in CD8⁺ T cell population (population size) mentioned above may be any measurable increase or elevation in the CD8⁺ T cell population (e.g. in the tumour microenvironment), e.g. as compared with a control level or value. Preferably, the level is significantly increased, compared to the level found in an appropriate control sample or subject. More preferably, the significantly increased levels are statistically significant, preferably with a probability value of <0.05 or <0.01 or <0.0025 or <0.0010 or 0.0001. In some embodiments, the increase may be at least 2%, at least 5%, at least 10%, at least 12%, at least 15%, at least 20%, at least 30%, at least 40%, at least 50%, at least 75%, at least 100%, or more, in comparison with an appropriate control (e.g. 2-5%, 2-10%, 5-10%, 2-15%, 5-15%, 10-15%, 2-20%, 5-20%, 10-20%, 15-20% or more, or e.g. up to 15%, up to 20%, up to 30%, up to 40%, up to 50%, up to 100%, up to 500% or up to 1000% or more).

Immunogenic cell death (ICD) may also be characterised by release of one or more ICD associated factors (or increase in the release of one or more ICD associated factors), such as the release of the non-chromatin-binding nuclear protein high-mobility group box 1 (HMGB1), calreticulin and/or ATP. The release (or increase in the release) of the non-chromatin-binding nuclear protein high-mobility group box 1 (HMGB1) is a preferred characteristic of ICD. Immunogenic cell death may thus be characterised by the release (or increase in the release) of one or more DAMPs (Damage-associated molecular pattern). DAMPs include ATP and HMGB1. A person skilled in the art would be readily able to assess the release of ICD associated factors, for example HMGB1 or ATP (or other DAMPs). Release of the above-mentioned factors is of course typically release from cancer cells. The presence and/or level of ICD associated factors may be assessed by any appropriate means in any appropriate sample (e.g. a serum sample from a subject). A suitable method for assessing the release of ICD associated factors, such as HMGB1, is immunoblotting. The “increase” may be any measurable increase or elevation of the level of one or more ICD associated factors, e.g. when compared with a control level or control value. Preferably, the level is significantly increased, compared to the level found in an appropriate control sample or subject. More preferably, the significantly increased levels are statistically significant, preferably with a probability value of <0.05. An example of a control level is the level of the ICD associated factor in question pre-treatment (prior to first administration of the immunotoxin). Thus, a control level may be the level of the ICD associated factor in question in the same subject pre-treatment (prior to first administration of the immunotoxin). Appropriate control “values” could also be readily determined without running a control “sample” in every test, e.g. by reference to the range for normal subjects or “pre-treatment” subjects known in the art.

Any appropriate antibody (or antibody moiety or antibody component or antibody part) may be present in an immunotoxin in accordance with the present invention. Appropriate antibodies may be readily selected by a skilled person, for example on the basis of the antigen to be targeted. Typically of course, the antibody used will bind to an antigen that is expressed in or on (present in or on) cells of the cancer to be treated in accordance with the invention. Thus, the antigen to be targeted is an antigen that is typically associated with (or characteristic of) the cancer to be treated in accordance with the invention.

As will be understood by those in the art, the immunological binding reagents encompassed by the term “antibody” extends to any immunological binding agent that comprises an antigen binding domain, including polyclonal and monoclonal antibodies (monoclonal antibodies are preferred). The term “antibody” extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The term “fragment” as used herein refers to fragments of biological relevance, e.g. fragments that contribute to antigen binding, e.g. form part of the antigen binding site, and/or contribute to the functional properties of the antibody. Certain preferred fragments comprise a heavy chain variable region (V_{H} domain) and/or a light chain variable region (V_{L} domain) of the antibodies used in accordance with the invention.

Antibodies can be fragmented using conventional techniques. Antibody fragments and other fragments can also be synthesized by recombinant techniques or can be chemically synthesized. Techniques for producing antibody fragments are well known and described in the art.

The techniques for preparing and using various antibody-based constructs and fragments are well known in the art.

In the present invention, preferably the antibody component of an immunotoxin comprises at least one heavy chain variable region (variable region of a heavy chain of an antibody molecule, VH domain) which comprises 3 heavy chain CDRs (heavy chain complementarity determining regions) and at least one light chain variable region (variable region of a light chain of an antibody molecule, VL domain) which comprises 3 light chain CDRs (light chain complementarity determining regions).

The term “heavy chain complementarity determining region” (“heavy chain CDR”) as used herein refers to regions of hypervariability within the heavy chain variable region (V_{H} domain) of an antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “light chain complementarity determining region” (“light chain CDR”) as used herein refers to regions of hypervariability within the light chain variable region (V_{L} domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

In certain embodiments, the antibody or antibody fragment used in accordance with the present invention comprises all or a portion of a heavy chain constant region (e.g. all or a portion of an Fc domain), such as an IgG1, IgG2, IgG3, IgG4, IgA1, IgA2, IgE, IgM or IgD constant region. Preferably, the heavy chain constant region is an IgG1 heavy chain constant region, or a portion thereof. Furthermore, the antibody or antibody fragment can comprise all or a portion of a light chain constant region. Appropriate sequences for such constant regions are well known and documented in the art. When a full complement of constant regions from the heavy and light chains are included in the antibodies (e.g. two heavy chains and two light chains), such antibodies are typically referred to herein as “full length” antibodies or “whole” antibodies.

In preferred embodiments of the present invention, the antibody component of the immunotoxin is a “whole” antibody or a “full-length” antibody. Full length IgG antibodies are preferred, for example IgG1 antibodies.

Antibody molecules can be produced in vitro or in vivo by any appropriate means.

An “Fv” fragment is the minimum antibody fragment that contains a complete antigen-recognition and binding site. This region has a dimer of one heavy chain and one light chain variable domain in tight, non-covalent association. It is in this configuration that the three hypervariable regions (CDRs) of each variable domain interact to define an antigen-binding site on the surface of the V_{H}-V_{L} dimer. Collectively, the six hypervariable regions (CDRs) confer antigen-binding specificity to the antibody.

However, it is well documented in the art that the presence of three CDRs from the light chain variable domain and three CDRs from the heavy chain variable domain of an antibody is not necessary for antigen binding. Thus, constructs smaller than the above classical antibody fragment or constructs with fewer than 6 CDRs are known to be effective.

In some embodiments, the antibody component of the immunotoxin may be a human antibody or a humanised antibody, but preferably the antibody component is a non-human antibody or a non-humanized antibody. Thus, preferably the antibody component is not a human antibody. Thus, preferably the antibody component is not a humanized antibody. In some embodiments, the antibody component of the immunotoxin may be a chimeric antibody, but preferably the antibody component is not a chimeric antibody. In some embodiments, the antibody component of the immunotoxin may be a murine or a rat antibody (e.g. a whole murine or a whole rat antibody).

Preferably, the antibody component of the immunotoxin is a murine antibody. Particularly preferred are murine full-length antibodies (or murine whole antibodies).

The term “human” as used herein in connection with antibody molecules and binding proteins refers to antibodies and binding proteins having variable regions or domains (e.g., V_{H}, V_{L}, CDR or FR regions or domains) and, optionally, constant antibody regions or domains, isolated or derived from a human or a human repertoire, or derived from or corresponding to sequences found in humans or a human repertoire, e.g., in the human germline or somatic cells.

The term “murine” antibody as used herein in connection with antibody molecules and binding proteins refers to antibodies and binding proteins having variable regions or domains (e.g., V_{H}, V_{L}, CDR or FR regions or domains) and, optionally, constant antibody regions or domains, isolated or derived from a mouse or a mouse repertoire, or derived from or corresponding to sequences found in mice or a mouse repertoire, e.g., in the mouse germline or somatic cells.

The term “humanized antibodies” as used herein refers to antibodies from non-human species in which certain amino acids have been changed to better correspond with the amino acids typically present in human antibodies.

The term “chimeric antibody” as used herein refers to an antibody that has an antigen binding region (e.g. variable domains of the heavy and light chains, VH and VL) from one species fused with the constant domain (effector region) from another species. “Chimeric” antibodies include chimeric mouse-human antibodies

In preferred embodiments, the antibody component of the immunotoxin comprises all or part of (preferably all of) a heavy chain constant region (preferably a mouse or rat heavy chain constant region, more preferably a mouse heavy chain constant region).

In preferred embodiments of the present invention the antibody component of the immunotoxin comprises all or part of (preferably all of) an Fc domain (or Fc region). An Fc region can also be referred to as a “Fragment crystallizable” region. The Fc region (or Fc domain) can be considered as a tail region of an antibody that may interact with cell surface receptors called Fc receptors. The Fc region can also allow antibodies to activate the immune system. An Fc region (e.g. of an IgG antibody) is typically composed of two identical protein fragments (or portions), derived from the antibody's two heavy chains.

Preferably, the Fc region is a mouse Fc region or a rat Fc region. More preferably, the Fc region is a mouse Fc region.

Without wishing to be bound by theory, it is believed that non-humanized or non-human antibodies (e.g. murine antibodies such as full-length murine antibodies having an Fc region) are of particular use in the present invention as the non-humanized or non-human (foreign) nature of the antibody may contribute to the immune effect of the immunotoxins that the inventors have observed in human subjects. Typically, those skilled in the art have opted to use human or humanized antibodies in immunotoxins rather than murine antibodies due to, for example, concern about a possible HAMA (human anti-mouse antibody) response in humans.

In particularly preferred embodiments, the antibody component of the immunotoxin is an antibody that binds to EpCAM or an antibody that binds to mucin-1 (MUC-1). Such an antibody component of the immunotoxin is typically capable of binding to EpCAM or mucin-1 (MUC-1) on the surface of a cancer cell.

In a preferred embodiment, the antibody component of the immunotoxin is an antibody that binds to EpCAM. EpCAM (CD326) may also be referred to as EGP-2, 17-1A, HEA125, MK-1, GA733-2, EGP34, KSA, TROP-1, ESA, TACSTD1 or KS1/4 and is one of the first identified tumor-associated antigens.

Preferably, the antibody component of the immunotoxin that binds to EpCAM is a whole (or full-length) antibody (e.g. an IgG antibody such as an IgG₁ antibody or other antibody comprising an Fc region or domain). Preferably, the antibody component that binds to EpCAM is a whole (or full-length) non-humanized or non-human antibody. More preferably, the antibody component that binds to EpCAM is a whole (or full length) murine antibody (e.g. a murine IgG antibody such as a murine IgG₁ antibody, e.g. the antibody MOC31).

In a preferred embodiment, the antibody component is (or comprises) the MOC31 antibody, or an antibody that is substantially homologous thereto. The MOC31 antibody is particularly preferred. The MOC31 antibody is a murine antibody (an IgG₁ antibody having mouse variable regions and mouse constant regions) that binds to EpCAM.

The amino acid sequence of the heavy chain variable region (VH domain) of the MOC31 antibody is set forth in SEQ ID NO:1. The amino acid sequence of the light chain variable region (VL domain) of the MOC31 antibody is set forth in SEQ ID NO:2.

Amino acid sequence of MOC31 heavy chain variable
region.
SEQ ID NO: 1
QVKLQQSGPELKKPGETVKISCKASGYTFTNYGMNWVKQAPGKGLKWMGW
INTYTGESTYADDFKGRFAFSLETSASAAYLQINNLKNEDTATYFVARFA
IKGDYWGQGTTVTVSS
Amino acid sequence of MCO31 light chain variable
region.
SEQ ID NO: 2
DIVLTQSPFSNPVTLGTSASISCRSTKSLLHSNGITYLYWYLQKPGQSPQ
LLIYQMSNLASGVPDRFSSSGSGTDFTLRISRVEAEDVGVYYCAQNLEIP
RTFGGGTKLEIKR

In some embodiments, the antibody component of the immunotoxin comprises a heavy chain variable region that comprises the amino acid sequence of SEQ ID NO:1 or a sequence substantially homologous thereto (e.g. a sequence having at least 80% at least 85%, at least 90%, at least 95%, at least 98% or at least 99% identity thereto), and/or a light chain variable region that comprises the amino acid sequence of SEQ ID NO:2 or a sequence substantially homologous thereto (e.g. a sequence having at least 80% at least 85%, at least 90%, at least 95%, at least 98% or at least 99% identity thereto). In preferred embodiments the antibody component of the immunotoxin comprises an Fc domain (or Fc region). Preferably, the Fc domain is a mouse Fc domain, preferably the Fc domain of the MOC31 antibody.

In some embodiments, the antibody component of the immunotoxin comprises a heavy chain variable region that comprises the amino acid sequence of SEQ ID NO:1 or a sequence substantially homologous thereto (e.g. a sequence having at least 80% at least 85%, at least 90%, at least 95%, at least 98% or at least 99% identity thereto), and a light chain variable region that comprises the amino acid sequence of SEQ ID NO:2 or a sequence substantially homologous thereto (e.g. a sequence having at least 80% at least 85%, at least 90%, at least 95%, at least 98% or at least 99% identity thereto). In preferred embodiments the antibody component of the immunotoxin comprises an Fc domain (or Fc region). Preferably, the Fc domain is a mouse Fc domain, preferably the Fc domain of the MOC31 antibody.

In some embodiments, the antibody component of the immunotoxin comprises a heavy chain variable region that comprises the amino acid sequence of SEQ ID NO:1 and a light chain variable region that comprises the amino acid sequence of SEQ ID NO:2. In preferred embodiments the antibody component of the immunotoxin comprises an Fc domain (or Fc region). Preferably, the Fc domain is a mouse Fc domain, preferably the Fc domain of the MOC31 antibody.

In some embodiments, the antibody component of the immunotoxin comprises a heavy chain that comprises a heavy chain variable region as set forth in SEQ ID NO: 1 or a heavy chain variable region (VH) sequence substantially homologous thereto (e.g. a sequence having at least 80% at least 85%, at least 90%, at least 95%, at least 98% or at least 99% identity thereto), and/or a light chain that comprises a light chain variable region as set forth in SEQ ID NO:2 or a light chain variable region (VL) sequence substantially homologous thereto (e.g. a sequence having at least 80% at least 85%, at least 90%, at least 95%, at least 98% or at least 99% identity thereto). Preferably, the Fc domain is a mouse Fc domain, preferably the Fc domain of the MOC31 antibody. In some preferred embodiments, the antibody component of the immunotoxin comprises the heavy chain of the MOC31 antibody and/or the light chain of the MOC31 antibody.

In some embodiments, the antibody component of the immunotoxin comprises a heavy chain that comprises a heavy chain variable region as set forth in SEQ ID NO:1 or a heavy chain variable region (VH) sequence substantially homologous thereto (e.g. a sequence having at least 80% at least 85%, at least 90%, at least 95%, at least 98% or at least 99% identity thereto), and a light chain that comprises a light chain variable region as set forth in SEQ ID NO:2 or a light chain variable region (VL) sequence substantially homologous thereto (e.g. a sequence having at least 80% at least 85%, at least 90%, at least 95%, at least 98% or at least 99% identity thereto). Preferably, the Fc domain is a mouse Fc domain, preferably the Fc domain of the MOC31 antibody.

In some embodiments, the antibody component of the immunotoxin comprises a heavy chain that comprises a heavy chain variable region as set forth in SEQ ID NO:1 and a light chain that comprises a light chain variable region as set forth in SEQ ID NO:2. In some preferred embodiments, the antibody component of the immunotoxin comprises the heavy chain of the MOC31 antibody and the light chain of the MOC31 antibody.

In some embodiments, the antibody component of the immunotoxin comprises at least one heavy chain variable region that comprises three CDRs and at least one light chain variable region that comprises three CDRs, wherein said heavy chain variable region comprises:
(a) a variable heavy (VH) CDR1 of (i.e. from within) the MOC31 heavy chain variable region as set forth in SEQ ID NO:1 or a sequence substantially homologous thereto,
(b) a VH CDR2 of (i.e. from within) the MOC31 heavy chain variable region as set forth in SEQ ID NO:1 or a sequence substantially homologous thereto, and
(c) a VH CDR3 of (i.e. from within) the MOC31 heavy chain variable region as set forth in SEQ ID NO:1 or a sequence substantially homologous thereto; and/or (preferably “and”) wherein said light chain variable region comprises:
(d) a variable light (VL) CDR1 of (i.e. from within) the MOC31 light chain variable region as set forth in SEQ ID NO:2 or a sequence substantially homologous thereto,
(e) a VL CDR2 of (i.e. from within) the MOC31 light chain variable region as set forth in SEQ ID NO:2 or a sequence substantially homologous thereto, and
(f) a VL CDR3 of (i.e. from within) the MOC31 light chain variable region as set forth in SEQ ID NO:2 or a sequence substantially homologous thereto;
wherein preferably said substantially homologous sequence is a sequence containing 1, 2 or 3 amino acid substitutions compared to the given CDR sequence, or wherein said substantially homologous sequence is a sequence containing conservative amino acid substitutions of the given CDR sequence. In preferred embodiments the antibody component of the immunotoxin comprises an Fc domain (or Fc region). Preferably, the Fc domain is a mouse Fc domain, preferably the Fc domain of the MOC31 antibody.

In some embodiments, the antibody component of the immunotoxin comprises at least one heavy chain variable region that comprises three CDRs and at least one light chain variable region that comprises three CDRs, wherein said heavy chain variable region comprises:
(a) a variable heavy (VH) CDR1 of (i.e. from within) the MOC31 heavy chain variable region as set forth in SEQ ID NO:1,
(b) a VH CDR2 of (i.e. from within) the MOC31 heavy chain variable region as set forth in SEQ ID NO:1, and
(c) a VH CDR3 of (i.e. from within) the MOC31 heavy chain variable region as set forth in SEQ ID NO:1; and wherein said light chain variable region comprises:
(d) a variable light (VL) CDR1 of (i.e. from within) the MOC31 light chain variable region as set forth in SEQ ID NO:2,
(e) a VL CDR2 of (i.e. from within) the MOC31 light chain variable region as set forth in SEQ ID NO:2, and
(f) a VL CDR3 of (i.e. from within) the MOC31 light chain variable region as set forth in SEQ ID NO:2.

The MOC31 antibody is commercially available from a number of suppliers (e.g. Abcam, ThermoFisher, Ventana, Cell Marque and Leica Biosystems). The MOC31 antibody is also known in the academic literature (e.g. Andersson et al. (2009) and (2015)). MOC31 antibody sequences are also described in Roovers et al. (Roovers et al., British Journal of Cancer, 1998, 78(11), p 1407-1416)).

In another preferred embodiment, the antibody component of the immunotoxin is an antibody that binds to mucin-1 (MUC-1).

Preferably, the antibody component of the immunotoxin that binds to mucin-1 is a whole (or full-length) antibody (e.g. an IgG antibody such as an IgG₁ antibody or other antibody comprising an Fc region or domain). Preferably, the antibody component that binds to mucin-1 is a whole (or full-length) non-humanized or non-human antibody. More preferably, the antibody component that binds to mucin-1 is a whole (or full length) murine antibody (e.g. a murine IgG antibody such as a murine IgG₁ antibody, e.g. BM7).

In a preferred embodiment, the antibody component is (or comprises) the BM7 antibody, or an antibody that is substantially homologous thereto. The BM7 antibody is particularly preferred. The BM7 antibody is a murine antibody (an IgG₁ antibody having mouse variable regions and mouse constant regions) that binds to mucin-1. The BM7 antibody is known in the academic literature, for example in Brugger et al. (Brugger et al., J. Clin. Oncol., 1999, 17(5):1535-1544). The BM7 antibody may also be obtained from Dr S. Kaul of the Frauenklinik, Heidelberg, Germany.

In some embodiments, the antibody component of the immunotoxin comprises a heavy chain of the BM7 antibody or a sequence substantially homologous thereto (e.g. a sequence having at least 80% at least 85%, at least 90%, at least 95%, at least 98% or at least 99% identity thereto), and/or a light chain of the BM7 antibody or a sequence substantially homologous thereto (e.g. a sequence having at least 80% at least 85%, at least 90%, at least 95%, at least 98% or at least 99% identity thereto).

In some embodiments, the antibody component of the immunotoxin comprises a heavy chain of the BM7 antibody or a sequence substantially homologous thereto (e.g. a sequence having at least 80% at least 85%, at least 90%, at least 95%, at least 98% or at least 99% identity thereto), and a light chain of the BM7 antibody or a sequence substantially homologous thereto (e.g. a sequence having at least 80% at least 85%, at least 90%, at least 95%, at least 98% or at least 99% identity thereto).

In some embodiments, the antibody component of the immunotoxin comprises a heavy chain of the BM7 antibody and a light chain of the BM7 antibody.

In some embodiments, the antibody component of the immunotoxin comprises a heavy chain variable region (VH) of the BM7 antibody, or a heavy chain variable region (VH) sequence substantially homologous thereto (e.g. a sequence having at least 80% at least 85%, at least 90%, at least 95%, at least 98% or at least 99% identity thereto), and/or a light chain variable region (VL) of the BM7 antibody or a light chain variable region (VH) sequence substantially homologous thereto (e.g. a sequence having at least 80% at least 85%, at least 90%, at least 95%, at least 98% or at least 99% identity thereto). In preferred embodiments the antibody component of the immunotoxin comprises an Fc domain (or Fc region). Preferably, the Fc domain is a mouse Fc domain, preferably the Fc domain of the BM7 antibody.

In some embodiments, the antibody component of the immunotoxin comprises a heavy chain variable region (VH) of the BM7 antibody, or a heavy chain variable region (VH) sequence substantially homologous thereto (e.g. a sequence having at least 80% at least 85%, at least 90%, at least 95%, at least 98% or at least 99% identity thereto), and a light chain variable region (VL) of the BM7 antibody or a light chain variable region (VH) sequence substantially homologous thereto (e.g. a sequence having at least 80% at least 85%, at least 90%, at least 95%, at least 98% or at least 99% identity thereto). In preferred embodiments the antibody component of the immunotoxin comprises an Fc domain (or Fc region). Preferably, the Fc domain is a mouse Fc domain, preferably the Fc domain of the BM7 antibody.

In some embodiments, the antibody component of the immunotoxin comprises a heavy chain variable region (VH) of the BM7 antibody and a light chain variable region (VL) of the BM7 antibody. In preferred embodiments the antibody component of the immunotoxin comprises an Fc domain (or Fc region). Preferably, the Fc domain is a mouse Fc domain, preferably the Fc domain of the BM7 antibody.

In some embodiments, the antibody component of the immunotoxin comprises at least one heavy chain variable region that comprises three CDRs and at least one light chain variable region that comprises three CDRs, wherein said heavy chain variable region comprises:
(a) a variable heavy (VH) CDR1 of (i.e. from within) the BM7 heavy chain or a sequence substantially homologous thereto,
(b) a VH CDR2 of (i.e. from within) the BM7 heavy chain or a sequence substantially homologous thereto, and
(c) a VH CDR3 of (i.e. from within) the BM7 heavy chain or a sequence substantially homologous thereto; and/or (preferably “and”) wherein said light chain variable region comprises:
(d) a variable light (VL) CDR1 of (i.e. from within) the BM7 light chain or a sequence substantially homologous thereto,
(e) a VL CDR2 of (i.e. from within) the BM7 light chain or a sequence substantially homologous thereto, and
(f) a VL CDR3 of (i.e. from within) the BM7 light chain or a sequence substantially homologous thereto;

wherein preferably said substantially homologous sequence is a sequence containing 1, 2 or 3 amino acid substitutions compared to the given CDR sequence, or wherein said substantially homologous sequence is a sequence containing conservative amino acid substitutions of the given CDR sequence. In preferred embodiments the antibody component of the immunotoxin comprises an Fc domain (or Fc region). Preferably, the Fc domain is a mouse Fc domain, preferably the Fc domain of the BM7 antibody.

In some embodiments, the antibody component of the immunotoxin comprises at least one heavy chain variable region that comprises three CDRs and at least one light chain variable region that comprises three CDRs, wherein said heavy chain variable region comprises:
(a) a variable heavy (VH) CDR1 of the BM7 heavy chain,
(b) a VH CDR2 of the BM7 heavy chain, and
(c) a VH CDR3 of the BM7 heavy chain; and wherein said light chain variable region comprises:
(d) a variable light (VL) CDR1 of the BM7 light chain,
(e) a VL CDR2 of the BM7 light chain, and
(f) a VL CDR3 of the BM7 light chain.

Preferred light chains, light chain variable regions and CDR regions for use in conjunction with the specified heavy chains, heavy chain variable regions and heavy chain CDR regions are described herein. However, other light chains or light chain variable regions that comprise three CDRs for use in conjunction with the specified heavy chains or heavy chain variable regions are also contemplated. Appropriate light chains or light chain variable regions which can be used in combination with the heavy chains or heavy chain variable regions described herein and which give rise to an antibody which binds to a relevant antigen (e.g. EpCAM or mucin-1) can be readily identified by a person skilled in the art.

For example, a preferred heavy chain or heavy chain variable region (e.g. of MOC31 or BM7) can be combined with a single light chain or light chain variable region or a repertoire of light chains or light chain variable regions and the resulting antibodies tested for binding to a relevant antigen.

If desired, similar methods could be used to identify alternative heavy chains or heavy chain variable regions for use in combination with preferred light chains or light chain variable regions.

In some embodiments, the antibody component of the immunotoxin is an antibody that binds to EpCAM and has the ability to compete with (i.e. bind to the same or substantially the same epitope as) the MOC31 antibody.

In some embodiments, the antibody component of the immunotoxin is an antibody that binds to MUC-1 and has the ability to compete with (i.e. bind to the same or substantially the same epitope as) the BM7 antibody.

Binding to the same epitope/antigen can be readily tested by methods well known and described in the art, e.g. using binding assays such as a competitive inhibition assay. Thus, a person skilled in the art will appreciate that binding assays can be used to identify other antibodies and antibody fragments with the same binding specificities as the antibodies and antibody fragments used in accordance with the invention.

Immunotoxins (or components thereof) used in accordance with the invention are generally “isolated” or “purified” molecules insofar as they are distinguished from any such components that may be present in situ within a human or animal body or a tissue sample derived from a human or animal body. The sequences may, however, correspond to or be substantially homologous to sequences as found in a human or animal body. Thus, the term “isolated” or “purified” as used herein in reference to immunotoxins (or components thereof) refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

Thus, when used in connection with immunotoxins or antibodies or toxic molecules used in accordance with the invention, the term “isolated” or “purified” typically refers to molecules that are substantially free of cellular material or other proteins from the source from which it is derived. In some embodiments, particularly where the molecule is to be administered to humans or animals, such isolated or purified proteins are substantially free of culture medium when produced by recombinant techniques, or chemical precursors or other chemicals when chemically synthesized.

The term “substantially homologous” as used herein in connection with an amino acid sequence includes sequences having at least 65%, 70% or 75%, preferably at least 80%, and even more preferably at least 85%, 90%, 95%, 96%, 97%, 98% or 99%, sequence identity to the amino acid sequences disclosed. Substantially homologous sequences of the invention thus include single or multiple base or amino acid alterations (additions, substitutions, insertions or deletions) to the sequences herein. At the amino acid level, preferred substantially homologous sequences contain up to 5, e.g. only 1, 2, 3, 4 or 5, preferably 1, 2 or 3, more preferably 1 or 2, altered amino acids, in one or more of the framework regions and/or one or more of the CDRs making up the sequences of the invention. In some embodiments, altered amino acids (e.g. amino acid substitutions) are not in the CDRs. Thus in some embodiments altered amino acids (e.g. amino acid substitutions) are in the framework regions and/or constant regions. Said alterations can be with conservative or non-conservative amino acids. Preferably said alterations are conservative amino acid substitutions.

Routine methods in the art such as alanine scanning mutagenesis and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

The term “substantially homologous” also includes modifications or chemical equivalents of the amino acid sequences described herein that perform substantially the same function as the proteins used in accordance with the invention in substantially the same way. For example, any substantially homologous antibody should retain the ability to bind to the relevant antigen, such as EpCAM or mucin-1, as described above. Preferably, any substantially homologous antibody should retain one or more (or all) of the functional capabilities of the starting antibody.

Preferably, any substantially homologous antibody should retain the ability to specifically bind to the same epitope of the relevant antigen, such as EpCAM or mucin-1, as recognized by the antibody in question, for example, the same epitope recognized by the CDR domains herein or the VH and VL domains herein or the heavy chain and light chain herein. Thus, preferably, any substantially homologous antibody should retain the ability to compete with the unmodified (unaltered) antibody for binding to a relevant antigen. For example, any antibody that is substantially homologous to the MOC31 antibody should retain the ability to compete with the MOC31 antibody for binding to EpCAM. Any antibody that is substantially homologous to the BM7 antibody should retain the ability to compete with the BM7 antibody for binding to mucin-1. Binding to the same epitope/antigen can be readily tested by methods well known and described in the art, e.g. using binding assays, e.g. a competition assay. Retention of other functional properties can also readily be tested by methods well known and described in the art or herein.

Thus, a person skilled in the art will appreciate that binding assays can be used to test whether “substantially homologous” antibodies have the same binding specificities as the antibodies and antibody fragments used in immunotoxins in accordance with the invention, for example, binding assays such as competition assays or ELISA assays. BIAcore assays could also readily be used to establish whether “substantially homologous” antibodies can bind to the relevant antigen, e.g. EpCAM or mucin-1. The skilled person will be aware of other suitable methods and variations.

An exemplary competition assay involves assessing the binding of various effective concentrations of an antibody to a relevant antigen (e.g. EpCAM or mucin-1) in the presence of varying concentrations of a test antibody (e.g. a substantially homologous antibody). The amount of inhibition of binding induced by the test antibody can then be assessed. A test antibody that shows increased competition with a reference antibody (e.g. MOC31 or BM7) at increasing concentrations (i.e. increasing concentrations of the test antibody result in a corresponding reduction in the amount of reference antibody binding to the relevant antigen) is evidence of binding to substantially the same epitope. Preferably, the test antibody significantly reduces the amount of reference antibody that binds to the relevant antibody. ELISA and/or flow cytometry assays may be used for assessing inhibition of binding in such a competition assay but other suitable techniques would be well known to a person skilled in the art.

In some embodiments, “substantially homologous” antibodies which retain the ability to specifically bind to substantially the same (or the same) epitope of the relevant antigen as recognized by the starting antibody are preferred. In some embodiments, antibodies which have the ability to compete with the MOC31 antibody are preferred. In some embodiments, antibodies which have the ability to compete with the BM7 antibody are preferred.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to a relevant antibody. Preferably, the competing antibody can bind to the same epitope as the reference antibody. Alternatively viewed, the competing antibody preferably has the same epitope specificity as the reference antibody (e.g. as MOC31 or BM7).

As the identification of competing antibodies is determined in comparison to a reference antibody, it will be understood that actually determining the epitope to which either or both antibodies bind is not in any way required in order to identify a competing antibody. However, epitope mapping can be performed using standard techniques, if desired.

Substantially homologous sequences of antibodies include, without limitation, conservative amino acid substitutions, or for example alterations that do not affect the VH, VL or CDR domains of the antibodies, e.g. antibodies where tag sequences, toxins or other components are added that do not contribute to the binding of antigen, or alterations to convert one type or format of antibody molecule or fragment to another type or format of antibody molecule or fragment (e.g. conversion from Fab to scFv or whole antibody or vice versa), or the conversion of an antibody molecule to a particular class or subclass of antibody molecule (e.g. the conversion of an antibody molecule to IgG or a subclass thereof, e.g. IgG₁).

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine).

Homology may be assessed by any convenient method. However, for determining the degree of homology between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CABIOS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having 65%, 70%, 75%, 80%, 85%, 90%, 95%, 96%, 97%, 98% or 99% homology, sequence identity etc. may be determined using the ALIGN program with default parameters (for instance available on Internet at the GENESTREAM network server, IGH, Montpellier, France).

The antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

A person skilled in the art will appreciate that the immunotoxins (and antibody and toxic moiety components thereof) may be prepared in any of several ways well known and described in the art. For example, a monoclonal antibody may be prepared using any technique that provides for the production of antibody molecules by continuous cell lines in culture. A toxic biological molecule may, for example, be isolated from the fermentation broth of a relevant microorganism. The antibody and toxic biological molecule may be linked (conjugated) by any appropriate linkage, e.g. a covalent bond (such as a thioether bond) as described elsewhere herein.

As discussed above, immunotoxins comprise a toxic (cytotoxic) component (or toxic moiety). The toxic moiety is a toxic biological molecule (a cytotoxic biological molecule). Typically, the toxic biological molecule is a protein (or peptide). Preferably, the toxic biological molecule is from bacteria (is a bacterial biological toxic molecule or is based on a bacterial toxic molecule), preferably the toxic biological molecule is a bacterial protein (or bacterial peptide). Thus, preferably the amino acid sequence of a bacterial toxic protein that is present in an immunotoxin corresponds to the amino acid sequence of the bacterial toxic protein as it occurs in nature (or is a variant thereof e.g. a substantially homologous variant, as described elsewhere herein). Preferably, the toxic biological molecule is an exotoxin, preferably a bacterial exotoxin. Particularly preferably, the toxic biological molecule is a bacterial exotoxin of bacteria of the genus Pseudomonas (is a Pseudomonas exotoxin or has an amino acid sequence that corresponds to a Pseudomonas exotoxin), more preferably of the species Pseudomonas aeruginosa such as Pseudomonas aeruginosa PA103. Particularly preferably, the toxic biological molecule is Pseudomonas exotoxin A (also referred to herein as PE).

The amino acid sequence of Pseudomonas exotoxin A is described in Gray et al., 1984 (Gray et al. 1984, Proc. Natl. Acad. Sci. USA, Vol. 81, p 2645-2649). The amino acid sequence of Pseudomonas exotoxin A is also set forth in Genbank Accession number AAB59097 (Version: AAB59097.1). In Genbank Accession number AAB59097, the sequence includes the signal peptide at amino acid residues 1 to 25, with the mature exotoxin A protein beginning at amino acid position 26 (residues 26 to 638). In accordance with the present invention, it is typically the mature exotoxin A protein (full-length, or wild-type, mature Pseudomonas exotoxin A) that is used in the immunotoxin (i.e. lacking the signal peptide). The sequence of mature full-length (wildtype) Pseudomonas exotoxin A is also set forth below as SEQ ID NO: 3.

Amino acid sequence of mature full-length
(wild-type) Psuedomonas exotoxin A.
SEQ ID NO: 3
AEEAFDLWNECAKACVLDLKDGVRSSRMSVDPAIADTNGQGVLHYSMVLE
GGNDALKLAIDNALSITSDGLTIRLEGGVEPNKPVRYSYTRQARGSWSLN
WLVPIGHEKPSNIKVFIHELNAGNQLSHMSPIYTIEMGDELLAKLARDAT
FFVRAHESNEMQPLTLAISHAGVSVVMAQTQPRREKRWSEWASGKVLCLL
DPLDGVYNYLAQQRCNLDDTWEGKIYRVLAGNPAKHDLDIKPTVISHRLH
FPEGGSLAALTAHQACHLPLETFTRHRQPRGWEQLEQCGYPVQRLVALYL
AARLSWNQVDQVIRNALASPGSGGDLGEAIREQPEQARLALTLAAAESER
FVRQGTGNDEAGAANADVVSLTCPVAAGECAGPADSGDALLERNYPTGAE
FLGDGGDVSFSTRGTQNWTVERLLQAHRQLEERGYVFVGYHGTFLEAAQS
IVFGGVRARSQDLDAIWRGFYIAGDPALAYGYAQDQEPDARGRIRNGALL
RVYVPRSSLPGFYRTSLTLAAPEAAGEVERLIGHPLPLRLDAITGPEEEG
GRLETILGWPLAERTVVIPSAIPTDPRNVGGDLDPSSIPDKEQAISALPD
YASQPGKPPREDLK

In some embodiments of the present invention, the toxic biological molecule is a protein (preferably a bacterial protein, more preferably Pseudomonas exotoxin A) that is essentially full-length. By “full-length” is meant that the protein is the same length (in terms of the number of amino acid residues) as the usual length (or wild-type or native length) of the protein (e.g. the same length as the protein as it may occur in nature for example in the bacteria from which it is derived or isolated or to which it corresponds). Thus, by “full-length” is meant “wild-type”. The term “essentially full-length” includes variants of a “full-length” sequence which may have one or more (e.g. 1, or up to 2, up to 3, up to 4, up to 5, up to 10, up to 20, up to 30, up to 40, up to 50 or up to 60) additional amino acids or one or more (e.g. 1, or up to 2, up to 3, up to 4, up to 5, up to 10, up to 20, up to 30, up to 40, up to 50 or up to 60) fewer amino acids as compared to the full-length (or wild-type or native length or usual length) of the protein. Thus, “essentially full-length” toxic moieties may be truncated and/or extended versions of the “full-length” counterpart. They may be truncated or extended at one or both termini (N terminus and/or C terminus). They may be truncated at one terminus and extended at the other. “Essentially full-length” toxic moieties may also contain an insertion of one or more amino acids at an internal position within the protein.

In some embodiments, “essentially full-length” variants are variants of a “full-length” sequence which have a length (in terms of the number of amino acid residues) that is at least 90% or at least 95% or at least 98% or at least 99% of the length of the “full-length” counterpart. Thus, in some embodiments, “essentially full-length” variants are variants of a “full-length” sequence which have a length that is at least 90% or at least 95% or at least 98% or at least 99% of the length of SEQ ID NO:3.

In some embodiments, the toxic biological molecule is a protein (e.g. a bacterial protein) that is a fragment of a full-length (or of an essentially full-length) counterpart (e.g. a fragment of Pseudomonas exotoxin A). Fragments include those molecules that are shorter than the “essentially full-length” molecules as discussed above.

However, in accordance with the present invention, preferably the toxic biological molecule is not a fragment. In particular, preferably the toxic biological molecule is not a fragment of Pseudomonas exotoxin A. Particularly preferably, the toxic biological molecule is not a fragment of Pseudomonas exotoxin A consisting of amino acid residues 252-608 of Pseudomonas exotoxin A. Preferably, the toxic biological molecule is not truncated with respect to the wild-type (or full-length or native) molecule.

In preferred embodiments, the toxic biological molecule is a toxic protein that comprises a cell-binding domain. In some preferred embodiments, the toxic biological molecule is a Pseudomonas exotoxin A that contains a cell-binding domain.

Of course, any fragments of toxic biological molecules or essentially full-length biological molecules (or otherwise modified toxic biological molecules) should have (or retain) cytotoxic activity (preferably substantially the same as, comparable to, or the same as the cytotoxic activity of a “full-length” or “wild-type” molecule). Whether or not cytotoxic activity is present (or retained) can be assessed by any appropriate means. Purely by way of example, a cell viability test could be employed (for example where the molecule in question is tested as a component in an immunotoxin), for example on HT29, SW480 or HCT116 cells. An exemplary cell viability assay is described in the Example section herein.

In preferred embodiments of the present invention, the toxic biological molecule comprises a full-length (or wild-type) protein. Preferably, the toxic biological molecule consists of a full-length (or wild-type) protein. Preferably, the toxic biological molecule comprises (or consists of) full-length (or wild-type) Pseudomonas exotoxin A. Particularly preferably, the toxic biological molecule consists of full-length (or wild-type) Pseudomonas exotoxin A. Thus, preferably, the toxic biological molecule consists of full-length Pseudomonas exotoxin A as set forth in SEQ ID NO: 3.

In some embodiments, the toxic biological molecule may be modified, for example, with respect to the form of the protein as it may occur in nature for example in the bacteria from which is derived or isolated (e.g. by one or more amino acid substitutions or deletions, for example e.g. 1, up to 2, up to 3, up to 4, up to 5, up to 10, up to 20, up to 30, up to 40 or up to 50 amino acid substitutions or deletions). Toxic proteins that may be used in accordance with the present invention include toxic biological molecules that are “substantially homologous” to full-length or to essentially full-length proteins. The term “substantially homologous” is discussed elsewhere herein.

However, in preferred embodiments of the present invention, the toxic biological molecule is not modified (is unmodified), e.g. in relation to a wild-type (or full-length or native) molecule. “Unmodified” means that there is no alteration in amino acid sequence as compared to a wild-type sequence (or full-length or native sequence). Thus, in some preferred embodiments, the toxic biological molecule (e.g. Pseudomonas exotoxin A) is a wild-type molecule.

Without wishing to be bound by theory, it is believed that the presence of an essentially full-length (or wild-type) toxic biological moiety (preferably a full-length or wild-type or unmodified toxic biological moiety) is particularly advantageous in the context of the present invention as it is believed to be important for the immunogenic effect of the immunotoxins. In this regard, it is believed that as an essentially full-length toxic biological moiety (preferably a full-length or unmodified toxic biological moiety) would be in its natural (or wild-type) conformation it would be more toxic (than fragments of a toxic biological moiety or modified forms) to cancer cells once it has been internalised. Also, it is believed that the essentially full-length (or full-length or unmodified) toxic moieties (e.g. full-length Pseudomonas exotoxin A) exhibit increased immunogenicity as they have not been modified (engineered) to reduce immunogenecity.

In many prior art immunotoxins, the toxic moiety (e.g. a PE-based moiety) is typically heavily truncated or modified. This is often done in an effort to reduce the immunogenicity of the toxic moiety and/or in an effort to decrease the size of the toxic moiety to increase penetration into a tumour. In accordance with the present invention, such truncated or modified versions of PE are typically not preferred.

Pseudomonas exotoxin A (PE) has three major structural domains which each have a function. These functions are the binding of cells or cell recognition (structural domain I, particularly structural domain 1a), translocation (to translocate the toxin across a cellular membrane—structural domain II), and ADP-ribosylation (structural domain III and a portion of structural domain 1b). In many PE-containing immunotoxins the PE moiety is truncated at a protease site between domains I and II. In preferred embodiments of the present invention, the toxic moiety is a Pseudomonas exotoxin A (PE) moiety that comprises structural domains I (preferably structural domains 1a and 1b), II and III. Thus, truncated (or otherwise modified) forms are typically not preferred.

In some preferred embodiments, the toxic biological molecule part of the immunotoxin is full-length (or wild-type or unmodified) Pseudomonas exotoxin A and the antibody part is a non-humanized or non-human, antibody (preferably a murine antibody). In some such embodiments, the toxic biological molecule part (or component) is conjugated to the antibody part (or component) by a thioether bond (e.g. as described elsewhere herein).

In some preferred embodiments, the toxic biological molecule part of the immunotoxin is full-length (wild-type) Pseudomonas exotoxin A and the antibody part binds to EpCAM and is a non-humanized or non-human, antibody (preferably a murine antibody). Preferably, the toxic biological molecule part of the immunotoxin is essentially full-length Pseudomonas exotoxin A and the antibody part is the MOC31 antibody that binds to EpCAM. Preferably, the toxic biological molecule part of the immunotoxin is full-length Pseudomonas exotoxin A and the antibody part is the MOC31 antibody that binds to EpCAM. In some embodiments, the toxic biological molecule part (or component) is conjugated to the antibody part (or component) by a thioether bond (e.g. as described elsewhere herein).

Preferably, the immunotoxin is MOC31PE. The immunotoxin MOC31PE has the MOC31 antibody conjugated to full-length (wild-type) Pseudomonas exotoxin A via a covalent (thioether) bond(s). Such thioether bonds may be formed (or created) using a sulfo-SMCC method or a SMCC method, e.g. as described elsewhere herein.

In some such embodiments, the conjugation (or linkage or attachment) between the MOC31 antibody molecule and the full-length (wild-type) Pseudomonas exotoxin A is a conjugation (or linkage or attachment) produced (or obtained) by contacting (or reacting) MOC31 antibody that has been treated (or derivatised or reduced) using (or with) DTT (dithiothreitol) to generate one or more free sulfhydryl (SH) groups on the antibody with full-length (wild-type) Pseudomonas exotoxin A that has been treated (or derivatised) using (or with) the SMCC (succinimidyl-4-(N-maleimidomethyl) cyclohexane-1-carboxylate) reagent. The conjugation (or attachment) is via a thioether bond(s) (via the sulphur atom of a sulfhydryl group(s)). Such a thioether bond may be formed on the free sulfhydryl group(s) after a hinge disulphide bond(s) of the antibody has been reduced by DTT. Thus, the full-length (wild-type) Pseudomonas exotoxin A may be attached to the MOC31 antibody at the position of a disulphide bond(s) in the hinge region of the antibody.

In other such embodiments, the conjugation (or linkage or attachment) between the MOC31 antibody molecule and the full-length (wild-type) Pseudomonas exotoxin A is a conjugation (or linkage or attachment) produced (or obtained) by contacting (or reacting) full-length (wild-type) Pseudomonas exotoxin A that has been treated (or derivatised or reduced) using (or with) DTT (dithiothreitol) to generate one or more free sulfhydryl (SH) groups on the full-length (wild-type) Pseudomonas exotoxin A with MOC31 antibody that has been treated (or derivatised) using (or with) the SMCC (succinimidyl-4-(N-maleimidomethyl) cyclohexane-1-carboxylate) reagent. The MOC31 antibody molecule and the full-length (wild-type) Pseudomonas exotoxin A are thus conjugated via a thioether bond(s) (via the sulphur atom of a sulfhydryl group(s)).

In some preferred embodiments, the toxic biological molecule part of the immunotoxin is full-length (wild-type) Pseudomonas exotoxin A and the antibody part binds to MUC-1 and is a non-humanized or non-human, antibody (preferably a murine antibody). Particularly preferably, the toxic biological molecule part of the immunotoxin is essentially full-length Pseudomonas exotoxin A and the antibody part is the BM7 antibody that binds to MUC-1. Particularly preferably, the toxic biological molecule part of the immunotoxin is full-length Pseudomonas exotoxin A and the antibody part is the BM7 antibody that binds to MUC-1. In some embodiments, the toxic biological molecule part (or component) is conjugated to the antibody part (or component) by a thioether bond (e.g. as described elsewhere herein).

Preferably, the immunotoxin is BM7PE. The immunotoxin BM7PE has the BM7 antibody conjugated to full-length (wild-type) Pseudomonas exotoxin A via a covalent (thioether) bond(s). Such thioether bonds may be formed (or created) using a sulfo-SMCC method or a SMCC method, e.g. as described elsewhere herein.

In some such embodiments, the conjugation (or linkage or attachment) between the BM7 antibody molecule and the full-length (wild-type) Pseudomonas exotoxin A is a conjugation (or linkage or attachment) produced (or obtained) by contacting (or reacting) BM7 antibody molecule that has been treated (or derivatised or reduced) using (or with) DTT (dithiothreitol) to generate one or more free sulfhydryl (SH) groups on the antibody molecule with full-length (wild-type) Pseudomonas exotoxin A that has been treated (or derivatised) using (or with) the SMCC (succinimidyl-4-(N-maleimidomethyl) cyclohexane-1-carboxylate) reagent. The conjugation (or attachment) is via a thioether bond(s) (via the sulphur atom of a sulfhydryl group(s)). Such a thioether bond may be formed on the free sulfhydryl group(s) after a hinge disulphide bond(s) of the antibody has been reduced by DTT. Thus, the full-length (wild-type) Pseudomonas exotoxin A may be attached to the BM7 antibody at the position of a disulphide bond(s) in the hinge region of the antibody.

In other such embodiments, the conjugation (or linkage or attachment) between the BM7 antibody molecule and the full-length (wild-type) Pseudomonas exotoxin A is a conjugation (or linkage or attachment) produced (or obtained) by contacting (or reacting) a full-length (wild-type) Pseudomonas exotoxin A that has been treated (or derivatised or reduced) using (or with) DTT (dithiothreitol) to generate one or more free sulfhydryl (SH) groups on the full-length (wild-type) Pseudomonas exotoxin A with BM7 antibody that has been treated (or derivatised) using (or with) the SMCC (succinimidyl-4-(N-maleimidomethyl) cyclohexane-1-carboxylate) reagent. The BM7 antibody molecule and the full-length (wild-type) Pseudomonas exotoxin A are thus conjugated via a thioether bond(s) (via the sulphur atom of a sulfhydryl group(s)).

Other toxic biological molecules that may be included in immunotoxins in accordance with the invention include diphtheria toxin, gelonin, ricin, shiga toxin, saponin and bouganin (or variants thereof of the type discussed elsewhere herein).

Toxic biological molecules for inclusion in immunotoxins may be prepared in any appropriate way and a person skilled in the art is familiar with suitable techniques for doing so. For example, bacterial protein toxins (e.g. exotoxins), such as Pseudomonas exotoxin A may be isolated from (or extracted from) a fermentation broth of Pseudomonas aeruginosa, such as Pseudomonas aeruginosa PA103. An appropriate and preferred method for isolating (or extracting) Pseudomonas exotoxin A from such a fermentation broth is described in the Example section. Thus, in some embodiments, Pseudomonas exotoxin A produced (or obtainable) by the fermentation of Pseudomonas aeruginosa, such as Pseudomonas aeruginosa PA103 (preferably as described in the Example section) may be used as the toxic biological molecule in the immunotoxin. Toxic biological molecules can be conjugated to the antibody part by any appropriate means. Alternatively, immunotoxins can be prepared by recombinant means.

As mentioned above, in the present invention, immunotoxins are used for treating cancer.

Of course, typically, the cancer to be treated will express (i.e. be positive for) the antigen (antigenic protein) to which the antibody part of the immunotoxin binds. Such an antigen may be expressed (or present or displayed) at the surface of the cancer cell.

In some preferred embodiments, the cancer is EpCAM-positive.

In some preferred embodiments the cancer is MUC-1-positive (mucin-1 positive). In some such embodiments the MUC-1 is a glycosylated form of MUC-1. Carcinomas often have aberrant MUC-1 glycosylation.

In some embodiments the cancer is a carcinoma or a sarcoma.

A preferred type of cancer is carcinoma (such as adenocarcinoma).

In some preferred embodiments, the cancer is metastatic cancer, preferably metastatic carcinoma. Preferably, the metastasis is not (or is not only) micrometastasis.

In some embodiments, the metastasis is micrometastasis.

In some embodiments, the cancer is a refractory cancer, preferably a refractory metastatic cancer (e.g. a refractory metastatic carcinoma such as colorectal carcinoma). Thus, the cancer may be a cancer that failed first-line or second-line treatment prior to being treated with an immunotoxin (e.g. MOC31PE) in accordance with the present invention. A refractory cancer is one which does not adequately respond to (or is resistant to) a cancer treatment.

In some embodiments, the cancer (e.g. a metastatic and/or advanced cancer) is a cancer that has failed one or more (e.g. 1, 2, 3 or 4, or all available, or all existing) therapies.

In some embodiments, the metastatic cancer is a progressive metastatic cancer (e.g. progressive metastatic colorectal cancer).

In some embodiments, the cancer is an advanced cancer (e.g. an advanced metastatic cancer).

In some embodiments, the cancer is an advanced metastatic cancer (e.g. an advanced metastatic carcinoma).

Preferably, the cancer is a solid (or bulky) tumour, particularly preferably a solid tumour that is a carcinoma. In some embodiments, the cancer is a large solid tumour (e.g. large carcinoma).

In some preferred embodiments, the cancer (e.g. a solid tumour such as a carcinoma) has its own blood supply. Preferably the cancer is a solid tumour (e.g. a carcinoma) that has its own blood supply. Thus preferably, the cancer is a tumour that is large enough to have its own blood supply. Thus, in some preferred embodiments the cancer (preferably a solid tumour such as a carcinoma) has developed (or is developing) its own blood supply. Thus, in some embodiments, the cancer (preferably a solid tumour such as a carcinoma) is served by one or more blood vessels. Thus, preferably the cancer (preferably a solid tumour such as a carcinoma) is connected to one or more blood vessels.

Alternatively viewed, the cancer (preferably a solid tumour such as a carcinoma) is preferably a vascularized tumour. Thus, the tumour may be of the size and/or dimensions that requires a blood supply (or vascularization) in order for it to be maintained and/or to grow and/or to spread. Whether or not a cancer has its own blood supply (or is vascularised) may be determined by any appropriate means, for example by imaging methods that are known in the art (e.g. ultrasound, CT (computed tomography) perfusion imaging, MRI-based imaging, functional PET (positron emission tomography) imaging or imaging based on the expression of markers (e.g. VEGF, VEGF receptors, integrins or matrix metalloproteinases) that are highly expressed on the endothelium of tumor vascalature).

Without wishing to be bound by theory, solid tumours (e.g. carcinomas, such as advanced and/or metastatic carcinomas) are a particularly attractive type of cancer to target with an immunotoxin in accordance with the present invention. In this regard, the immunotoxin does not need to penetrate an entire solid tumour (e.g. a carcinoma) and exert its cytotoxic effect directly on all cells in the tumour (which can be difficult or impossible for solid tumours particularly large solid tumours e.g a tumour which is large enough to have its own blood supply or vasculature), but rather the immunotoxin can target a limited number of cancer cells and induce immunogenic cell death and the subject's immune response can be relied upon to target the remainder of the tumour. The limited number of cancer cells may be cancer cells that are at, or close to, the tumour surface, and thus are typically accessible to the immunotoxin.

Thus, typically the cancer is not a haematological cancer.

Cancers, or cell proliferative disorders, that may be treated in accordance with the present invention include sarcomas and carcinomas (preferably human sarcomas or carcinomas), including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma.

In some embodiments, if the cancer is a sarcoma, osteosarcoma and chondrosarcoma are preferred.

In some embodiments the cancer, or cell proliferative disorder that that may be treated in accordance with the present invention include leukemias, acute lymphocytic leukemia and acute myelocytic leukemia (myeloblastic, promyelocytic, myelomonocytic, monocytic and erythroleukemia), chronic leukemia (chronic myelocytic (granulocytic) leukemia and chronic lymphocytic leukemia), polycythemia vera, lymphoma (Hodgkin's disease and non-Hodgkin's disease), multiple myeloma, Waldenstrom's macroglobulinemia, and heavy chain disease.

In some preferred embodiments, the cancer is selected from the group consisting of colorectal cancer, ovarian cancer, renal cancer, breast cancer, glioblastoma, malignant melanoma, prostate cancer, pancreatic cancer, lung cancer (e.g. non-small cell lung cancer), gastric cancer and bladder cancer. Metastatic forms of these cancers are particularly preferred. Advanced forms of these cancers are also particularly preferred.

In particularly preferred embodiments, the cancer is colorectal cancer, preferably metastatic colorectal cancer. In some embodiments, the cancer is an advanced colorectal cancer (e.g. an advanced metastatic colorectal cancer).

In some embodiments, the cancer is ovarian cancer, preferably metastatic ovarian cancer.

In some embodiments, the cancer is breast cancer, preferably metastatic breast cancer, more preferably triple-negative breast cancer.

In some embodiments, the cancer is bladder cancer or gastric cancer.

In preferred embodiments, the cancer is selected from the group consisting of colorectal carcinoma, ovarian carcinoma, renal carcinoma, breast carcinoma, glioblastoma, malignant melanoma, prostate carcinoma, pancreatic carcinoma, lung carcinoma, gastric carcinoma and bladder carcinoma.

In a particularly preferred embodiment the cancer is colorectal carcinoma. In some preferred embodiments the cancer is an EpCAM-positive colorectal carcinoma. Preferably, the cancer is metastatic colorectal carcinoma. Preferably, the cancer is EpCAM-positive metastatic colorectal carcinoma. In some such embodiments, preferably the immunotoxin is MOC31 PE.

In a preferred embodiment the cancer is colorectal carcinoma. In some preferred embodiments the cancer is a MUC-1-positive colorectal carcinoma. Preferably, the cancer is metastatic colorectal carcinoma. Preferably, the cancer is MUC-1-positive metastatic colorectal carcinoma. In some such embodiments, preferably the immunotoxin is BM7PE.

In some embodiments, the cancer is selected from the group consisting of renal cancer, malignant melanoma and gastric cancer. Metastatic forms of these cancers are particularly preferred. In some embodiments, the cancer is selected from the group consisting of renal carcinoma, malignant melanoma, and gastric carcinoma.

In some embodiments, the cancer is a MUC-1+ haematological cancer (e.g. myeloma). In some embodiments, the cancer is an EpCAM+ sarcoma.

In some embodiments, the cancer is not a mucinous peritoneal surface malignancy.

Any stage of cancer may be treated in accordance with the present invention. “Stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

Staging of cancer can also be based on the revised criteria of TNM staging by the American Joint Committee for Cancer (AJCC) published in 1988. Staging is the process of describing the extent to which cancer has spread from the site of its origin. It is used to assess a patient's prognosis and to determine the choice of therapy. The stage of a cancer is determined by the size and location in the body of the primary tumor, and whether it has spread to other areas of the body. Staging involves using the letters T, N and M to assess tumors by the size of the primary tumor (T); the degree to which regional lymph nodes (N) are involved; and the absence or presence of distant metastases (M)—cancer that has spread from the original (primary) tumor to distant organs or distant lymph nodes. Each of these categories is further classified with a number 1 through 4 to give the total stage. Once the T, N and M are determined, a “stage” of I, II, III or IV is assigned. Stage I cancers are small, localized and usually curable. Stage II and III cancers typically are locally advanced and/or have spread to local lymph nodes. Stage IV cancers usually are metastatic (have spread to distant parts of the body) and generally are considered inoperable. In some embodiments, Stage III or Stage IV cancers are preferred, preferably stage IV cancers.

In preferred embodiments, immunotoxins used in accordance with the invention provide a sustained anti-cancer effect. Without wishing to be bound by theory, induction of immunogenic cell death by immunotoxins in accordance with the present invention can provide a sustained anti-cancer effect. In this regard, some immunotoxins have a very short half-life in the body which means, if they act by purely by a direct effect on protein synthesis (inhibition of protein synthesis) and the induction of apoptosis, their operational lifespan in the body can be very short and thus the anti-cancer effect is not a sustained effect. For example, the antibody MOC31 has a half-life of about 3 hours. However, immunotoxins that can induce immunogenic cell death of cancer cells can provide an indirect, longer lasting (or longer acting or longer term), anti-cancer effect (longer lasting than a direct effect on apoptosis or protein synthesis), as once the anti-cancer immune response is induced, the anti-cancer effect can persist (be maintained) even once the immunotoxin itself has been cleared (is no longer present). This provides a sustained (or long lasting) anti-cancer effect and can prolong a subject's survival, e.g. prolong overall survival. Such an indirect, sustained, effect via immunogenic cell death can be beneficial, for example fewer and/or less frequent doses and/or lower doses of the immunotoxin may be necessary to achieve the anti-cancer effect (as compared to an immunotoxin only acting via a direct effect).

Also, again without wishing to be bound by theory, as immunogenic cell death involves the raising of an immune response against the cancer cells, there may be an immunological memory meaning that if the cancer were to return or metastasize at a future time point, the immune system could recognise the cancer cells and raise an immune response against the cancer. Thus, in some embodiments, immunotoxin treatments in accordance with the present invention may be used in subjects at risk of cancer relapse or recurrence or metastasis. Thus, alternatively viewed, in some embodiments, immunotoxins are used in the prevention of cancer relapse or recurrence or metastasis. Further alternatively viewed, in some embodiments the immunotoxin protects (e.g. provides long-term protection) against cancers that recur and/or metastasize. Thus, the immunotoxin may provide anti-cancer or anti-tumour immunity, e.g. provide long-term protection against cancer relapse, recurrence and/or metastasis.

In some cancer treatments in accordance with the present invention, the immunotoxin treatment may be combined with a further active agent (e.g. a further anti-cancer agent).

However, in some preferred embodiments of the present invention the immunotoxin is used as the sole active agent (sole active agent in the treatment regimen). Thus, in some preferred embodiments the treatment is a monotherapy. Monotherapy refers to the use of a single drug to treat a disease or condition, in this case cancer. Thus, in some preferred embodiments the immunotoxin is used alone. By “sole active agent” (or sole active ingredient) is meant the sole agent or ingredient that is therapeutically active (or biologically active). Thus, components such as preservatives or excipients or agents that are not relevant to the disease being treated are not considered to be active agents.

In preferred embodiments, the treatment regimen does not include any non-antibody based active agents. Alternatively viewed, in some embodiments, the treatment regimen only includes antibody based active agents (e.g. only includes an immunotoxin)

Particularly preferably, cancer treatments in accordance with the present invention are done in the absence of an immunosuppressor (absence of an agent that suppresses or inhibits the ability of the subject being treated to raise an immune response). In a particularly preferred embodiment, cancer treatments in accordance with the present invention are done in the absence of the immunosuppressor cyclosporine A (CsA, e.g. Sandimmune®). Put another way, preferably the treatment regimen does not include an immunosuppressor.

As mentioned above, immunotoxins may preferably be used as the sole active agent. However, in some embodiments, the immunotoxin is combined with one or more further (additional) active agents. For example, in some embodiments, a cancer therapy in accordance with the present invention may be characterised by the administration of an immunostimulant (an agent that stimulates or enhances a subject's immune system or immune response). Thus, in some embodiments a combination of an immunotoxin and an immunostimulant may be used to treat cancer.

An immunostimulant may be, speaking generally, administered to a subject substantially simultaneously with the immunotoxin, such as from a single pharmaceutical composition or from two pharmaceutical compositions administered closely together (at the same or a similar time). Alternatively, an immunostimulant may be administered to a subject at a time prior to or sequential to the administration of the immunotoxin. “At a time prior to or sequential to”, as used herein, means “staggered”, such that an immunostimulant is administered to a subject at a time distinct to the administration of the immunotoxin. Generally, the two agents may be administered at times effectively spaced apart to allow the two agents to exert their respective therapeutic effects, i.e., they are administered at “biologically effective time intervals”. In some embodiments, an immunostimulant is administered as part of the same therapeutic regimen as the immunotoxin.

In some preferred embodiments, the immunostimulant is an agent that targets T-cells, either directly or indirectly, and preferably stimulates (or contributes to) a T cell immune response (e.g. a Th1 immune response).

In some preferred embodiments, the immunostimulant is a checkpoint inhibitor (or an immune checkpoint inhibitor). Checkpoint inhibitors are a well-known class of anti-cancer agents which can promote immune activity, e.g. in a tumour microenvironment. A checkpoint inhibitor is a type of drug that blocks (or inhibits) certain proteins on some types of immune system cells, such as T cells, or some cancer cells. These proteins usually help suppress immune responses and can help prevent T cells from killing cancer cells. When these proteins are blocked (or inhibited) by checkpoint inhibitors, the immune system can be released from an inhibited state and T cells are better able to kill cancer cells. Given that immunotoxins used in accordance with the invention can induce immunogenic cell death of cancer cells, it is important that subjects being treated are able to raise an immune response (have an active immune system). Thus, combination treatments with immunostimulants (e.g. checkpoint inhibitors) would be beneficial as they promote immune activity.

In some preferred embodiments, the checkpoint inhibitor is (or comprises) an antibody, for example an antibody (e.g. a monoclonal antibody) that binds to PD-1, PD-L1, CTLA-4 or B7-H3. Antibodies (e.g. monoclonal antibodies) that bind to PD-1, PD-L1 or CTLA-4 are typically preferred immune checkpoint inhibitory antibodies.

In some embodiments, the checkpoint inhibitor is selected from the group consisting of Nivolumab, Pembrolizumab, Ipilimumab, Atezolizumab, Durvalumab, Avelumab and Cemiplimab.

Nivolumab is a PD-1 inhibitor (Bristol Myers Squibb, brand name Opdivo). Pembrolizumab is a PD-1 inhibitor (Merck Sharp & Dohme, brand name Keytruda). Ipilimumab is a CTLA-4 inhibitor (Bristol Myers Squibb, brand name Yervoy). Atezolizumab is a PD-L1 inhibitor (Roche, brand name Tecentriq). Durvalumab is a PD-L1 inhibitor (Astra Zeneca, brand name Imfinzi). Avelumab is a PD-L1 inhibitor (Pfizer and Merck KGaA, brand name Bavencio). Cemiplimab is a PD-1 inhibitor (Sanofi and Regeneron, preliminary brand name REGN-2810).

In some embodiments, the checkpoint inhibitor may be a non-antibody based molecule, e.g. a small molecule inhibitor (e.g. that inhibits PD-1, PD-L1, CTLA-4 or B7-H3).

In some embodiments, the immunostimulant is an agent (e.g. an antibody) that targets a protein selected from the group consisting of IDO, OX40 and 4-1 BB. In some embodiments, the immunostimulant is an inhibitor (e.g. an antibody) of IDO, OX40 or 4-1BB.

In other embodiments, the immunostimulant is GM-CSF or C-CSF. The immunostimulant may also be an interferon, an interleukin or a chemotherapeutic drug.

Subjects treated in accordance with the present invention are preferably humans. In some embodiments, human subjects have an ECOG performance status of 0-2. Veterinary treatments (e.g. for cows, sheep, pigs, dogs, cats, horses) are also contemplated. Typically of course, subjects in accordance with the present invention are subjects having cancer. Preferred cancer types are described elsewhere herein. For example, as indicated elsewhere herein, in some embodiments the subject is a subject having metastatic cancer. In some embodiments the subject is a subject having advanced cancer (e.g. advanced metastatic cancer).

In preferred embodiments, subjects treated in accordance with the present invention have an active (or functioning) immune system, more preferably a fully active (or fully functioning) immune system. Accordingly, preferably subjects being treated in accordance with the present invention are capable of raising an effective immune response (e.g. a T-cell response such as a Th1 response). Thus, preferably, subjects are typically and preferably not exposed to (and preferably have not been exposed to) any immunosuppressive agent (e.g. cyclosporine A, CsA) as it is believed that such subjects will not be capable of induction of an effective ICD response. Thus, preferably, subjects are typically and preferably not taking (and preferably have not taken) an immunosuppressive agent. Preferably subjects have not been otherwise diagnosed as having an impaired immune system (or being immunodeficient). Preferred subjects thus do not have an impaired immune system (or are not immunodeficient) as it is believed that such subjects will not be capable of induction of an effective ICD response.

In some embodiments, a subject (preferably a human subject) is a subject that is refractory to, or ineligible for, standard chemotherapy.

In some embodiments, a subject (preferably a human subject) is a subject that is refractory to, or ineligible for, first-line or second-line treatment (e.g. standard chemotherapy). Thus, in some embodiments, the immunotoxin in accordance with the present invention may be used as a second-line or third-line therapy.

However, in some embodiments, the immunotoxin in accordance with the present invention may be used as a first-line therapy.

In some embodiments, the subject is a subject that has relapsed after a first-line treatment with another drug(s) (e.g. relapsed within two years of first-line treatment, e.g. with a 12-18 month progression-free survival). In some such embodiments, the subject has colorectal cancer or ovarian cancer or gastric cancer.

In some embodiments, the subject is a subject that has relapsed after a multiple lines of treatment with another drug(s). In some embodiments, the subject is a subject that has relapsed after second-line or third-line treatment with another drug(s). In some such embodiments, the subject has colorectal cancer or ovarian cancer or gastric cancer.

In some embodiments, the subject is a subject for which there is no alternative therapeutic option (e.g. no effective alternative therapeutic option).

In some embodiments, the subject is a subject that has been treated (i.e. previously treated) with one or more (e.g. 1, 2, 3 or 4, or more, or all available, or all existing) other anti-cancer therapies (e.g. non-immunotoxin based therapies, e.g. standard chemotherapy). In some embodiments, such other treatments have failed. Thus, in some embodiments, the subject is a subject in which one or more (e.g. 1, 2, 3 or 4, or more, or all available, or all existing) other anti-cancer therapies (e.g. non-immunotoxin based therapies, e.g. standard chemotherapy) have failed. In some embodiments, a first-line therapy has failed in the subject. In some embodiments, a first-line therapy and a second-line therapy have failed in the subject.

In some embodiments, the subject is not a subject with minimal residual disease (e.g. low or very low or negligible tumour burden). In some embodiments, the subject is not a subject with no (or low) evidence of disease but at high risk of relapse.

The term “treatment” or “therapy” includes any treatment or therapy which results in an improvement in the health or condition of a patient, or of a symptom of the cancer they are suffering. “Treatment” is not limited to curative therapies (e.g. those which result in the elimination of cancer cells or tumours or metastases from the patient), but includes any therapy which has a beneficial effect on the cancer or the patient, for example, tumour regression or reduction, reduction of metastatic potential, increased overall survival, extension or prolongation of life or remission, induction of remission, a slow-down or reduction of disease progression or the rate of disease progression, or of tumour development, subjective improvement in quality of life, reduced pain or other symptoms related to the disease, improved appetite, reduced nausea, or an alleviation of any symptom of the cancer.

Immunotoxins for use in the present invention may be included in formulations (or compositions). Such formulations may be for pharmaceutical or veterinary use. Suitable diluents, excipients and carriers for use in such formulations are known to the skilled person.

Immunotoxins (or formulations) for use in accordance with the present invention may be administered to a subject via any appropriate route.

The immunotoxins (or formulations) may be presented, for example, in a form suitable for oral, nasal, parenteral, intravenous, topical or rectal administration. Preferably, the compositions are presented in a form suitable for systemic (e.g. intravenous) administration.

The pharmaceutical compositions (formulations) may be administered parenterally. Parenteral administration may be performed by subcutaneous, intramuscular or intravenous injection, e.g. by means of a syringe. Alternatively, parenteral administration (e.g. i.v. infusion) can be performed by means of an infusion pump. Intraperitoneal (i.p.) administration or intratumoral administration (e.g. injection) may be used in some embodiments.

In some embodiments administration may be by intravesical instillation.

Administration may be systemic or local.

In preferred embodiments, immunotoxins are systemically administered. In particularly preferred embodiments the immunotoxins (or formulations) are administered intravenously (i.v.)

In some preferred embodiments, the administration is not local administration (for example not intraperitoneal (i.p.) administration or intratumoural administration).

In some embodiments the immunotoxin is both systemically and locally administered.

In some embodiments, if the cancer is bladder cancer, the administration may be local (or locoregional), for example by intravesical instillation.

In some preferred embodiments of the present invention, the subject is a human subject (preferably with an active immune system), the administration is systemic administration (preferably intravenous administration), and the cancer is a carcinoma (e.g. metastatic carcinoma such as metastatic colorectal carcinoma). In some embodiments, preferably the antibody component of the immunotoxin is a non-humanized or non-human, antibody (e.g. a murine antibody—preferably with an Fc domain—such as MOC31 or BM7) and the toxic moiety of the immunotoxin is Pseudomonas exotoxin A (preferably full-length or wildtype or unmodified Pseudomonas exotoxin A). In some preferred embodiments, the immunotoxin is MOC31PE or BM7PE.

The active compounds defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions are then filled into injection vials or ampoules.

Dosages, and dosage regimens, may vary based on parameters such as the age, weight and sex of the subject. Appropriate dosages can be readily established. In some embodiments, immunotoxin doses of 0.5, 1.0, 1.5, 2.0, 3.0, 4.0, 5.0, 6.0, 6.5, 7.0, 8.0, 9.0, 10, 15, 20, or 25 μg kg⁻¹ may be used (e.g. via intravenous infusion e.g. for about 20 mins, e.g. at one week, two week, three week, four week, five week or six week intervals e.g. up to 2, 3, 4, 5, 6, 7, 8, 9 or 10 times in total). In some embodiments, the immunotoxin dose may be up to 10 μg kg⁻¹ (e.g. via intravenous infusion). In some embodiments, immunotoxin (e.g. MOC31 PE) doses of 0.5, 1.0, 1.5, 2.0, 3.0, 4.0, 5.0, 6.0, 6.5, 7.0 or 8.0 μg kg⁻¹ may be used (e.g. via intravenous infusion e.g. for about 20 mins, e.g. at two week intervals e.g. up to four times in total). Appropriate dosage units can readily be prepared.

The pharmaceutical compositions for use in the present invention may additionally comprise further therapeutically active ingredients as described above in the context of co-administration regimens. However, as discussed elsewhere herein, in some preferred embodiments, in compositions for use in the present invention the immunotoxin is the sole active agent present.

In one preferred aspect, the present invention provides an immunotoxin for use in treating a carcinoma in humans, wherein the immunotoxin induces immunogenic cell death of tumour cells, wherein the immunotoxin comprises a murine antibody conjugated to a full-length Pseudomonas exotoxin A (PE), wherein said murine antibody comprises an Fc domain, and wherein said immunotoxin is systemically administered.

In preferred embodiments, said carcinoma is colon carcinoma.

In preferred embodiments, said carcinoma is a metastatic carcinoma, preferably a metastatic colon carcinoma.

In some preferred embodiments, said carcinoma is an EpCAM-positive carcinoma.

In some preferred embodiments, said carcinoma is a MUC-1-positive carcinoma.

In preferred embodiments, said murine antibody is a full-length antibody, preferably an IgG antibody, for example an IgG₁ antibody.

In preferred embodiments, said full-length Pseudomonas exotoxin A (PE) has the amino acid sequence set forth in SEQ ID NO: 3.

In some preferred embodiments, said murine antibody is an antibody that binds to EpCAM.

In some preferred embodiments, said murine antibody is the antibody MOC31.

In some preferred embodiments, said immunotoxin is MOC31 PE.

In some preferred embodiments, said murine antibody is an antibody that binds to MUC-1.

In some preferred embodiments, said murine antibody is the antibody BM7.

In some preferred embodiments, said immunotoxin is BM7PE.

In some preferred embodiments, said immunotoxin is the sole active agent used in the treatment regimen. In other preferred embodiments, said immunotoxin is used in combination with an immunostimulant, for example a checkpoint inhibitor.

In preferred embodiments, said immunotoxin is administered intravenously.

In one preferred aspect, the invention provides an immunotoxin for use in treating a carcinoma in humans, wherein the immunotoxin induces immunogenic cell death of tumour cells, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, and wherein said immunotoxin
(i) is used in combination with an immunostimulant; or
(ii) is the sole active agent used in the treatment regimen.

The present invention also provides a method of treating cancer, said method comprising administering to a subject in need thereof a therapeutically effective amount of an immunotoxin that induces immunogenic cell death of cancer cells. Embodiments of the uses of the invention described above apply, mutatis mutandis, to this aspect of the invention.

A therapeutically effective amount can be determined based on the clinical assessment and can be readily monitored.

The present invention also provides the use of an immunotoxin that induces immunogenic cell death of cancer cells in the manufacture of a medicament for treating cancer. Embodiments of the uses of the invention described above apply, mutatis mutandis, to this aspect of the invention.

The present invention also provides a method of treating a carcinoma in humans, said method comprising systemically administering to a human in need thereof a therapeutically effective amount of an immunotoxin that induces immunogenic cell death of cancer cells, wherein the immunotoxin comprises a murine antibody conjugated to a full-length Pseudomonas exotoxin A (PE), wherein said murine antibody comprises an Fc domain. Embodiments of the uses of the invention described above apply, mutatis mutandis, to this aspect of the invention.

In one aspect, the present invention provides a method of treating a carcinoma in humans, said method comprising systemically administering to a human in need thereof a therapeutically effective amount of an immunotoxin that induces immunogenic cell death of cancer cells, wherein the immunotoxin comprises a murine antibody conjugated to a full-length Pseudomonas exotoxin A (PE), wherein said murine antibody binds to EpCAM or to MUC-1 and comprises an Fc domain, and wherein said immunotoxin
(i) is used in combination with an immunostimulant; or
(ii) is the sole active agent used in the treatment regimen.

The present invention also provides the use of an immunotoxin that induces immunogenic cell death of cancer cells in humans when systemically administered, in the manufacture of a medicament for treating a carcinoma, wherein the immunotoxin comprises a murine antibody conjugated to a full-length Pseudomonas exotoxin A (PE), wherein said murine antibody comprises an Fc domain. Embodiments of the uses of the invention described above apply, mutatis mutandis, to this aspect of the invention.

In one aspect, the present invention provides the use of an immunotoxin that induces immunogenic cell death of cancer cells in humans when systemically administered, in the manufacture of a medicament for treating a carcinoma, wherein the immunotoxin comprises a murine antibody conjugated to a full-length Pseudomonas exotoxin A (PE), wherein said murine antibody binds to EpCAM or to MUC-1 and comprises an Fc domain, and wherein said immunotoxin
(i) is used in combination with an immunostimulant; or
(ii) is the sole active agent used in the treatment regimen.

In another aspect, the present invention provides an immunotoxin for use in treating cancer in humans, wherein the immunotoxin is the sole active agent used in the treatment regimen. Embodiments of the uses of the invention described elsewhere herein apply, mutatis mutandis, to this aspect of the invention.

In preferred embodiments of this aspect of the invention, the immunotoxin is systemically administered (preferably intravenously administered).

In some embodiments, the cancer is a carcinoma or a sarcoma, preferably a carcinoma.

In preferred embodiments, the human subject has an active (functional) immune system.

In preferred embodiments, the administration is systemic administration (preferably intravenous administration) and the cancer is a carcinoma (e.g. metastatic carcinoma such as metastatic colorectal carcinoma). In some embodiments, preferably the antibody component of the immunotoxin is a non-humanized or non-human, antibody (e.g. a murine antibody—preferably with an Fc domain—such as MOC31 or BM7) and the toxic moiety of the immunotoxin is Pseudomonas exotoxin A (preferably full-length or wild-type or unmodified Pseudomonas exotoxin A). In some preferred embodiments, the sole active agent is the immunotoxin MOC31PE or the immunotoxin BM7PE.

In preferred embodiments, the immunotoxin induces immunogenic cell death of cancer cells in the subject.

In one preferred aspect, the invention provides an immunotoxin for use in treating a carcinoma in humans, wherein the immunotoxin comprises a murine antibody conjugated to a full-length Pseudomonas exotoxin (PE), wherein said murine antibody comprises an Fc domain, wherein said immunotoxin is systemically administered, and wherein the immunotoxin is the sole active agent used in the treatment regimen.

In preferred embodiments, said carcinoma is colon carcinoma.

In preferred embodiments, said carcinoma is a metastatic carcinoma, preferably a metastatic colon carcinoma.

In some preferred embodiments, said carcinoma is an EpCAM-positive carcinoma

In some preferred embodiments, said carcinoma is a MUC-1-positive carcinoma.

In preferred embodiments, said murine antibody is a full-length antibody, preferably an IgG antibody, for example an IgG₁ antibody.

In preferred embodiments, said full-length Pseudomonas exotoxin A (PE) has the amino acid sequence set forth in SEQ ID NO: 3.

In some preferred embodiments, said murine antibody is an antibody that binds to EpCAM.

In some preferred embodiments, said murine antibody is the antibody MOC31. In some preferred embodiments, said immunotoxin is MOC31PE.

In some preferred embodiments, said murine antibody is an antibody that binds to MUC-1.

In some preferred embodiments, said murine antibody is the antibody BM7.

In some preferred embodiments, said immunotoxin is BM7PE.

In preferred embodiments, said immunotoxin is administered intravenously.

In one preferred aspect, the present invention provides an immunotoxin for use in treating a carcinoma in humans, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, and wherein the immunotoxin is the sole active agent used in the treatment regimen.

The present invention also provides a method of treating cancer, said method comprising administering to a subject in need thereof a therapeutically effective amount of an immunotoxin wherein the immunotoxin is the sole active agent used in the treatment regimen. Embodiments of the uses of the invention described above apply, mutatis mutandis, to this aspect of the invention.

A therapeutically effective amount can be determined based on the clinical assessment and can be readily monitored.

The present invention also provides the use of an immunotoxin in the manufacture of a medicament for treating cancer wherein said immunotoxin is the sole active agent used in the treatment regimen. Embodiments of the uses of the invention described above apply, mutatis mutandis, to this aspect of the invention.

The present invention also provides a method of treating a carcinoma in humans, said method comprising systemically administering to a human in need thereof a therapeutically effective amount of an immunotoxin, wherein the immunotoxin is the sole active agent used in the treatment regimen, wherein the immunotoxin comprises a murine antibody conjugated to a full-length Pseudomonas exotoxin A (PE) and wherein said murine antibody comprises an Fc domain. Embodiments of the uses of the invention described above apply, mutatis mutandis, to this aspect of the invention.

In one preferred aspect, the present invention provides a method of treating a carcinoma in humans, said method comprising systemically administering to a human in need thereof a therapeutically effective amount of an immunotoxin, wherein the immunotoxin is the sole active agent used in the treatment regimen, wherein the immunotoxin comprises a murine antibody conjugated to a full-length Pseudomonas exotoxin A (PE) and wherein said murine antibody binds to EpCAM or to MUC-1 and comprises an Fc domain.

The present invention also provides the use of an immunotoxin in the manufacture of a medicament for treating a carcinoma in humans by systemic administration of the immunotoxin, wherein said immunotoxin is the sole active agent used in the treatment regimen, wherein the immunotoxin comprises a murine antibody conjugated to a full-length Pseudomonas exotoxin A (PE) and wherein said murine antibody comprises an Fc domain. Embodiments of the uses of the invention described above apply, mutatis mutandis, to this aspect of the invention.

In one preferred aspect, the present invention provides the use of an immunotoxin in the manufacture of a medicament for treating a carcinoma in humans by systemic administration of the immunotoxin, wherein said immunotoxin is the sole active agent used in the treatment regimen, wherein the immunotoxin comprises a murine antibody conjugated to a full-length Pseudomonas exotoxin A (PE) and wherein said murine antibody binds to EpCAM or to MUC-1 and comprises an Fc domain.

In another aspect, the present invention provides a combination of an immunotoxin and an immunostimulant for use in treating cancer in humans. Alternatively viewed, in another aspect, the present invention provides an immunotoxin for use in treating cancer in humans, wherein the therapeutic regimen further comprises the administration of an immunostimulant. An immunostimulant is an agent that stimulates or enhances a subject's immune system or immune response. Embodiments of the uses of the invention described elsewhere herein apply, mutatis mutandis, to these aspects of the invention.

In preferred embodiments, the immunotoxin is systemically administered (preferably intravenously administered).

In preferred embodiments, the human subject has an active (functional) immune system.

In some embodiments, the cancer is a carcinoma or a sarcoma, preferably a carcinoma.

In preferred embodiments, the immunotoxin induces immunogenic cell death of cancer cells in the subject.

In preferred embodiments, the administration is systemic administration (preferably intravenous administration) and the cancer is a carcinoma (e.g. metastatic carcinoma such as metastatic colorectal carcinoma). In some embodiments, preferably the antibody component of the immunotoxin is a non-humanized or non-human, antibody (e.g. a murine antibody—preferably with an Fc domain—such as MOC31 or BM7) and the toxic moiety of the immunotoxin is Pseudomonas exotoxin A (preferably full-length or wild-type or unmodified Pseudomonas exotoxin A). In some preferred embodiments, the immunotoxin is MOC31 PE or BM7PE. Preferred immunostimulants are described elsewhere herein and include immune checkpoint inhibitors (for example immune checkpoint inhibitory antibodies such as PD-1, PD-L1, CTLA-4 and B7-H3 antibodies). Antibodies (e.g. monoclonal antibodies) that bind to PD-1, PD-L1 or CTLA-4 are typically preferred immune checkpoint inhibitory antibodies. In some embodiments, the checkpoint inhibitor is selected from the group consisting of Nivolumab, Pembrolizumab, Ipilimumab, Atezolizumab, Durvalumab, Avelumab and Cemiplimab.

A combination of an immunotoxin and an immunostimulant does not necessarily mean that the immunotoxin and the immunostimulant are present in the same composition (although this is contemplated in some embodiments). Indeed, the immunotoxin and the immunostimulant may be administered separately (e.g. physically separate administration and/or temporally separate administration). As described elsewhere herein, an immunotoxin and an immunostimulant may be administered closely together (at the same or a similar time) or, alternatively, an immunostimulant may be administered to a subject at a time prior to or sequential to the administration of the immunotoxin. In some embodiments, an immunostimulant is administered as part of the same therapeutic regimen as the immunotoxin.

In one preferred aspect, the present invention also provides a combination of an immunotoxin and an immunostimulant for use in treating a carcinoma in humans, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered.

The present invention also provides a method of treating cancer, said method comprising administering to a subject in need thereof a combination of a therapeutically effective amount of an immunotoxin and an immunostimulant. The present invention provides a method of treating cancer, said method comprising administering to a subject in need thereof a therapeutically effective amount of an immunotoxin, wherein the therapeutic regimen further comprises the administration of an immunostimulant. Embodiments of the uses of the invention described above apply, mutatis mutandis, to these aspects of the invention.

A therapeutically effective amount can be determined based on the clinical assessment and can be readily monitored.

In one preferred aspect, the present invention provides a method of treating a carcinoma in humans, said method comprising systemically administering to a human in need thereof a combination of a therapeutically effective amount of an immunotoxin and an immunostimulant, wherein the immunotoxin comprises a murine antibody conjugated to a full-length Pseudomonas exotoxin A (PE) and wherein said murine antibody binds to EpCAM or to MUC-1 and comprises an Fc domain.

The present invention also provides the use of a combination of an immunotoxin and an immunostimulant in the manufacture of a medicament for treating cancer. The present invention also provides the use of an immunotoxin in the manufacture of a medicament for treating cancer, wherein the therapeutic regimen for the treatment of cancer further comprises the administration of an immunostimulant. Embodiments of the uses of the invention described above apply, mutatis mutandis, to these aspects of the invention.

In one preferred aspect, the present invention provides the use of a combination of an immunotoxin and an immunostimulant in the manufacture of a medicament for treating a carcinoma in humans by systemic administration of the immunotoxin, wherein the immunotoxin comprises a murine antibody conjugated to a full-length Pseudomonas exotoxin A (PE) and wherein said murine antibody binds to EpCAM or to MUC-1 and comprises an Fc domain.

In another aspect, the present invention provides an immunotoxin for use in stimulating (or enhancing or activating) the immune system in a subject. Alternatively viewed, the present invention provides an immunotoxin for use in stimulating (or eliciting) an immune response in a subject. Alternatively viewed, the present invention provides a method for enhancing the immune system (or stimulating an immune response) in a subject, said method comprising administering an immunotoxin to said subject. The present invention also provides a use of an immunotoxin in the manufacture of a medicament for enhancing the immune system in a subject (or stimulating an immune response in a subject). Embodiments of the uses of the invention described elsewhere herein apply, mutatis mutandis, to these aspects of the invention.

In preferred embodiments, the subject is a human subject (typically a human subject having cancer for example a cancer as described elsewhere herein). In preferred embodiments, the administration is systemic administration (preferably intravenous administration). Preferably the antibody component of the immunotoxin is a non-humanized or non-human, antibody (e.g. a murine antibody—preferably with an Fc domain—such as MOC31 or BM7) and the toxic moiety of the immunotoxin is Pseudomonas exotoxin A (preferably full-length Pseudomonas exotoxin A). In some preferred embodiments, the immunotoxin is MOC31PE. In some preferred embodiments, the immunotoxin is BM7PE.

Typically, the stimulation (or enhancement) of the immune system is the stimulation (or enhancement or activation) of a T-cell response (e.g. Th1 cytokine response). In some embodiments, the stimulation (or enhancement) of the immune system may be characterized by a cytokine response (e.g. a Th1 cytokine response) as defined elsewhere herein. Such a cytokine response may be determined by any appropriate means, for example by ELISA assay on a sample (e.g. a serum sample) from a subject (e.g. as described elsewhere herein). A particularly preferred assay is described in the Example section herein.

The stimulation (or enhancement) of the immune system (or of an immune response) may be characterized by an activation (or maturation) of dendritic cells in a subject being treated with the immunotoxin (e.g. as described elsewhere herein).

In one preferred aspect, the present invention provides an immunotoxin for use in stimulating (or enhancing or activating) the immune system in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

Alternatively viewed, the present invention provides a method for stimulating (or enhancing or activating) an immune response in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

Alternatively viewed, the present invention provides the use of an immunotoxin in the manufacture of a medicament for stimulating (or enhancing or activating) an immune response in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

The stimulation (or enhancement) of the immune system (or of an immune response) may result in cell death of tumour cells, e.g. immunogenic cell death. Thus, methods for inducing immunogenic cell death are also contemplated.

In one aspect, the present invention provides an immunotoxin for use in inducing (or enhancing or activating) immunogenic cell death in a subject. Embodiments of the uses of the invention described elsewhere herein apply, mutatis mutandis, to this aspect of the invention.

In one preferred aspect, the present invention provides an immunotoxin for use in inducing (or enhancing or activating) immunogenic cell death in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In another aspect, the present invention provides a method for inducing (or enhancing or activating) immunogenic cell death in a subject, said method comprising administering an immunotoxin to said subject. Embodiments of the uses of the invention described elsewhere herein apply, mutatis mutandis, to this aspect of the invention.

In one preferred aspect, the present invention provides a method for inducing (or enhancing or activating) immunogenic cell death in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In another aspect, the present invention provides the use of an immunotoxin in the manufacture of a medicament for inducing (or enhancing or activating) immunogenic cell death in a subject. Embodiments of the uses of the invention described elsewhere herein apply, mutatis mutandis, to this aspect of the invention.

In one preferred aspect, the present invention provides the use of an immunotoxin in the manufacture of a medicament for inducing (or enhancing or activating) immunogenic cell death in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In one aspect, the present invention provides an immunotoxin for use in inducing (or enhancing or increasing or activating) dendritic cell maturation in a subject. Embodiments of the uses of the invention described elsewhere herein apply, mutatis mutandis, to this aspect of the invention.

In one preferred aspect, the present invention provides an immunotoxin for use in inducing (or enhancing or increasing or activating) dendritic cell maturation in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In another aspect, the present invention provides a method for inducing (or enhancing or increasing or activating) dendritic cell maturation in a subject, said method comprising administering an immunotoxin to said subject. Embodiments of the uses of the invention described elsewhere herein apply, mutatis mutandis, to this aspect of the invention.

In one preferred aspect, the present invention provides a method for inducing (or enhancing or increasing or activating) dendritic cell maturation in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In another aspect, the present invention provides the use of an immunotoxin in the manufacture of a medicament for inducing (or enhancing or increasing or activating) dendritic cell maturation in a subject. Embodiments of the uses of the invention described elsewhere herein apply, mutatis mutandis, to this aspect of the invention.

In one preferred aspect, the present invention provides the use of an immunotoxin in the manufacture of a medicament for inducing (or enhancing or increasing or activating) dendritic cell maturation in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In one aspect, the present invention provides an immunotoxin for use in activating (or promoting or enhancing or increasing or inducing) a T cell response in a subject. In some embodiments, the T cell response is characterized by an increase in the CD8⁺ T cell population. Embodiments of the uses of the invention described elsewhere herein apply, mutatis mutandis, to this aspect of the invention.

In one preferred aspect, the present invention provides an immunotoxin for use in activating (or promoting or enhancing or increasing or inducing) a T cell response in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered. In preferred embodiments, said immunotoxin (i) is used in combination with an immunostimulant, or (ii) is the sole active agent used in the treatment regimen.

In another aspect, the present invention provides a method for activating (or promoting or enhancing or increasing or inducing) a T cell response in a subject, said method comprising administering an immunotoxin to said subject. Embodiments of the uses of the invention described elsewhere herein apply, mutatis mutandis, to this aspect of the invention.

In one preferred aspect, the present invention provides a method for activating (or promoting or enhancing or increasing or inducing) a T cell response in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In another aspect, the present invention provides the use of an immunotoxin in the manufacture of a medicament for activating (or promoting or enhancing or increasing or inducing) a T cell response in a subject. Embodiments of the uses of the invention described elsewhere herein apply, mutatis mutandis, to this aspect of the invention.

In one preferred aspect, the present invention provides the use of an immunotoxin in the manufacture of a medicament for activating (or promoting or enhancing or increasing or inducing) a T cell response in a human subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, In preferred embodiments, said immunotoxin (i) is used in combination with an immunostimulant, or (ii) is the sole active agent used in the treatment regimen.

In a further aspect, the present invention provides kits comprising one or more of the immunotoxins or formulations as defined above for use according to the invention. Preferred immunotoxins are described elsewhere herein. The kits may comprise further components. Each component may be provided in a separate compartment or vessel. Where convenient and practical, mixtures of components could be provided. The components may be provided in dry, e.g. crystallised, freeze dried or lyophilised, form or in solution, typically such liquid compositions will be aqueous and buffered with a standard buffer such as Tris, HEPES, etc.

Such kits may comprise further components (e.g. as described above).

Preferably the kits are for use in treating cancer, e.g. are for use in the methods or uses of the present invention as described herein.

In one aspect, the present invention provides a kit comprising an immunotoxin and an immunostimulant (immunostimulatory agent e.g. a checkpoint inhibitor).

The kits may also be provided with instructions for using the kit in accordance with the invention or with directions for how such instructions may be obtained.

The invention will be further described with reference to the following non-limiting Examples with reference to the following drawings in which:

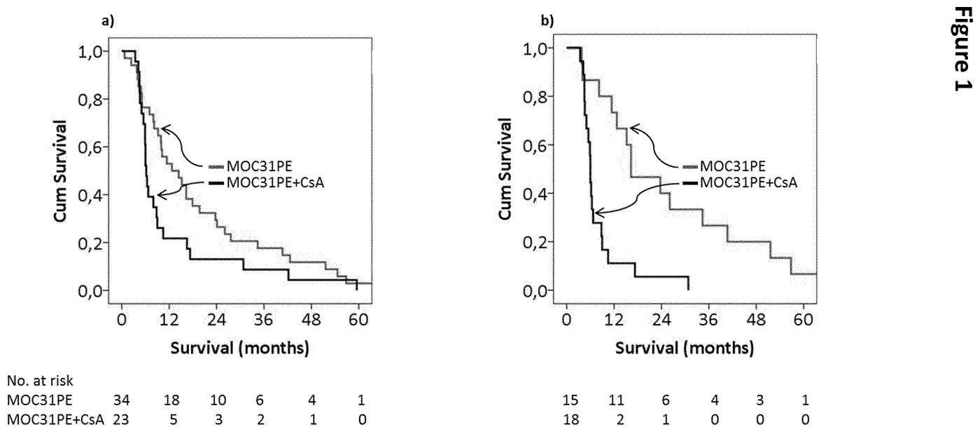

FIG. 1: Overall survival: Kaplan-Meier plot.

(a) Overall survival of patients with EpCAM-positive metastatic cancers treated with MOC31PE (n=34) or with MOC31PE plus Cyclosporin A (CsA, n=23), p<0.066 (b) Overall survival of patients with EpCAM-positive metastatic colorectal cancer treated with MOC31PE (n=15) or with MOC31PE plus CsA (n=18). The MOC31PE treated group and MOC31 PE plus CsA treated group are indicated by arrows, p<0.001. The significance of differences in survival between MOC31 PE and MOC31 PE plus CsA patients was determined by the log-rank test. Cum survival=cumulative survival.

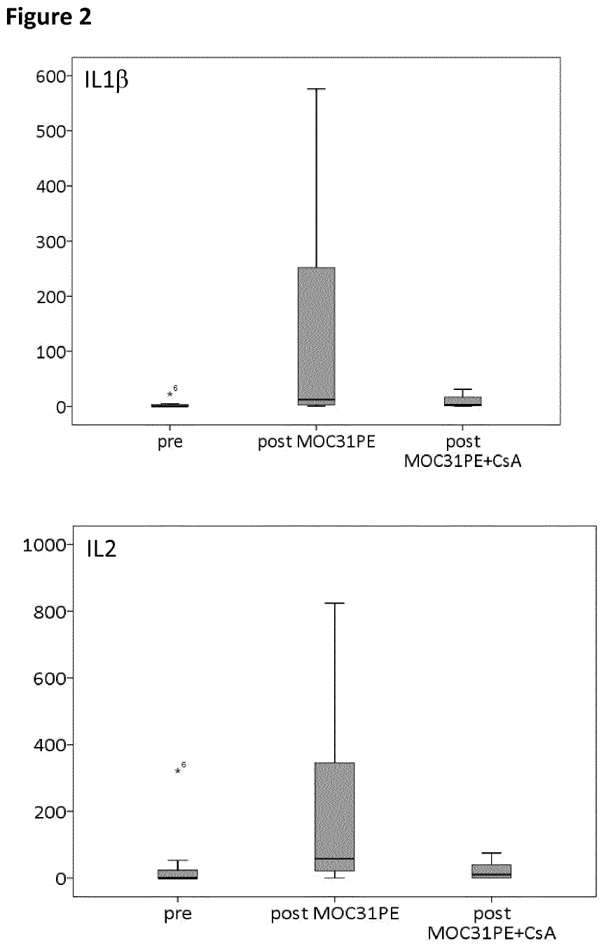

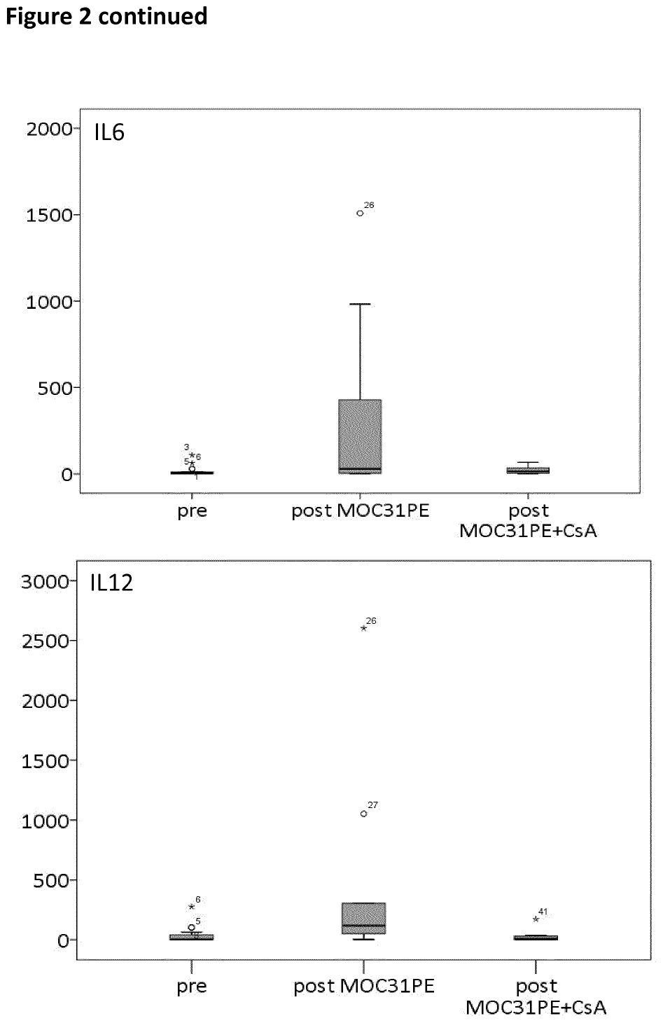

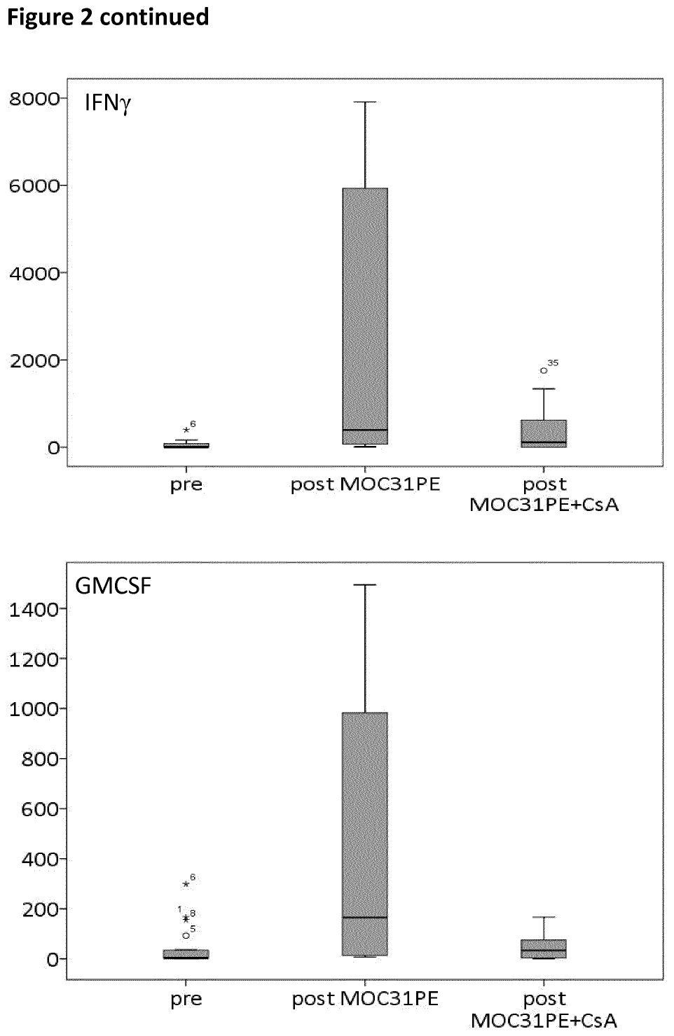

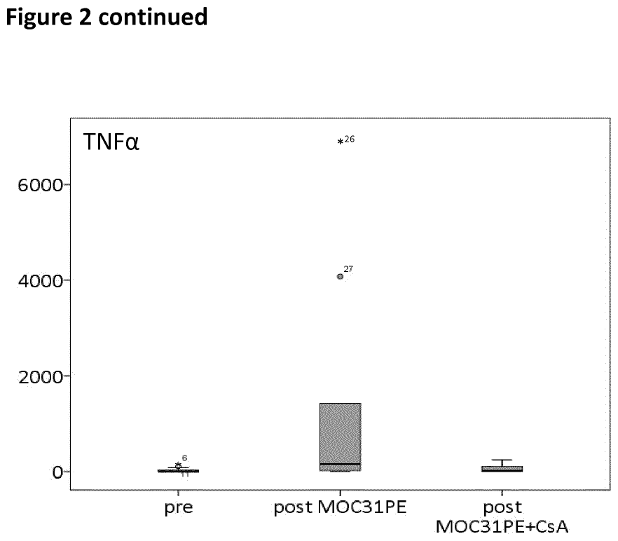

FIG. 2: Box plot showing the cytokine level in patient serum

The levels (pg/ml) of indicated cytokines in serum of metastatic colorectal cancer patients; pre-treatment (pre, n=20), two weeks after MOC31 PE (post MOC31PE, n=9) and MOC31PE plus CsA (post MOC31PE+CsA, n=12) treatment measured by multiplex cytokine ELISA assay. The box plots show median values (horizontal lines), interquartile ranges (the box lengths), extreme values (*) and outliers (o)

FIG. 3: MOC31PE induced cytotoxicity and HMGB1 release in colorectal cancer cell lines.

(a) HCT116, SW480 and HT29 cells were incubated with MOC31 PE (1-1000 ng/ml) for 24 h. Cell viability is expressed as a percentage (mean) of the value obtained in vehicle treated cells. Results are representative of 2 independent experiments, each plated in at least triplicate. (b) Western blot analysis of high mobility group box 1 protein (HMGB1) in supernatant from HCT116 cells, untreated (vehicle control), treated with MOC31 PE immunotoxin (10 and 100 ng/ml) and MOC31 monoclonal antibody (100 ng/ml) for 24 h. Equal volumes of supernatants were run on SDS-PAGE gel and stained with anti-HMGB1 antibody. Results are representative of 2 independent experiments. FIG. 3 (b) also shows western blot analysis of high mobility group box 1 protein (HMGB1) in supernatant from SW480 cells, untreated (vehicle control) and treated with MOC31PE immunotoxin (100 and 1000 ng/ml). Recombinant HMGB1 (with a tag) was included as a positive control in the western blot.

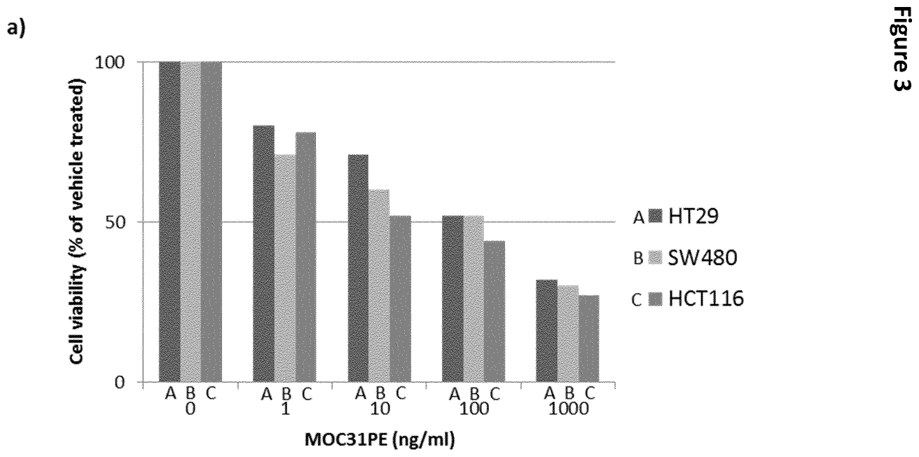

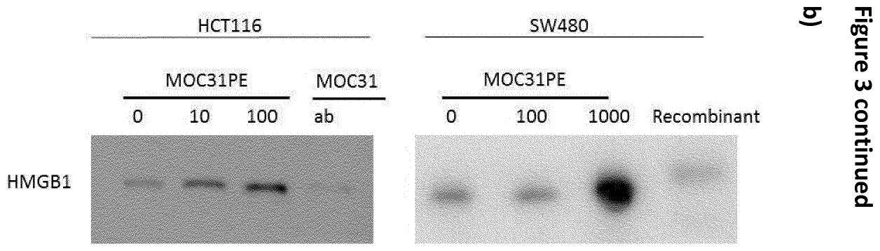

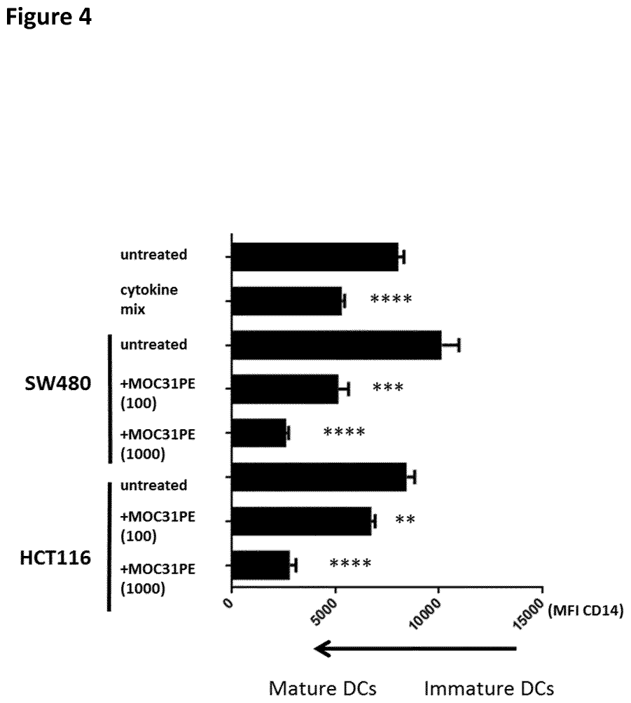

FIG. 4: Ex vivo maturation of immature dendritic cells (DCs) induced by MOC31PE.

Immature DCs treated with conditioned medium from MOC31PE treated colorectal cancer cells (HCT116 or SW480) show a significant decrease in CD14 expression compared to immature DCs+conditioned medium from non-treated cells (HCT116 or SW480). Mean fluorescence intensity (MFI) decrease in CD14 expression analyzed by flow cytometry. Concentrations of MOC31PE were as indicated, 100 or 1000 ng/ml. The result is representative of triplicates. **** p<0.0001, *** p<0.0010, ** p<0.0025.

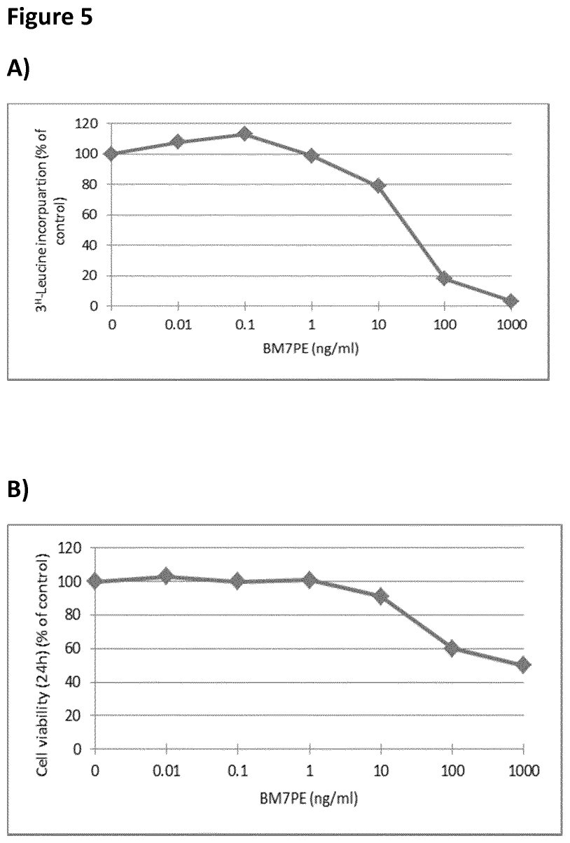

FIG. 5: FIG. 1: BM7PE induced cytotoxicity in breast cancer cell line.

(A) Inhibition of protein synthesis in Muc-1 expressing T47D breast cancer cells. BM7PE inhibits protein synthesis effectively in a dose-dependent way. (B) Cell viability in Muc-1 expressing T47D breast cancer cells. The cytotoxicity was measured after 24 hr treatment.

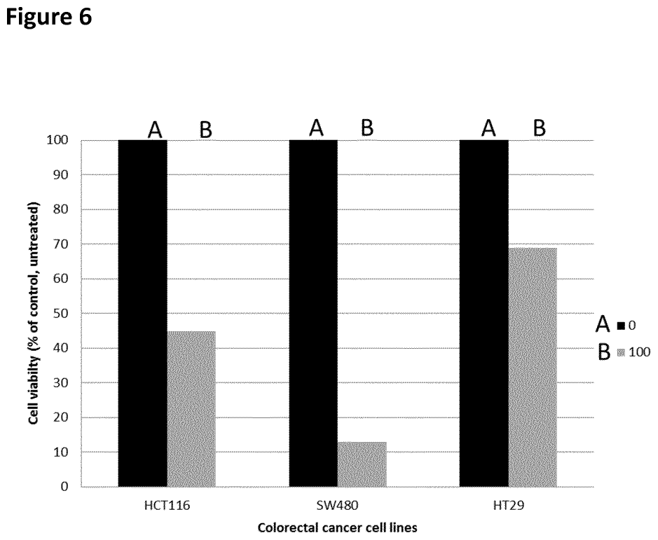

FIG. 6: BM7PE induced cytotoxicity in colorectal cancer cell line.

Cell viability in Muc-1 expressing HCT116, SW480 and HT29 colorectal cancer cells. Cells were incubated with BM7PE (100 ng/ml) for 48 h. Cell viability is expressed as a percentage (mean) of the value obtained in vehicle treated cells.

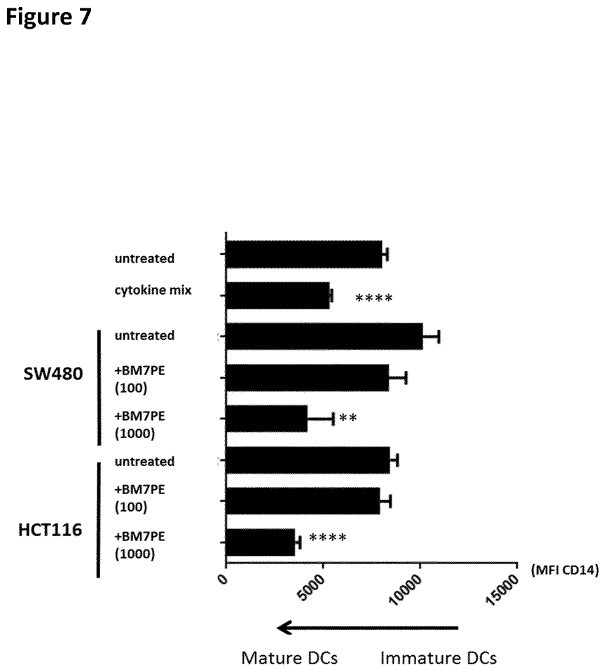

FIG. 7: Ex vivo maturation of immature dendritic cells (DCs) induced by BM7PE.

Immature DCs treated with conditioned medium from BM7PE treated colorectal cancer cells (HCT116 or SW480) show a significant decrease in CD14 expression compared to immature DCs+conditioned medium from non-treated cells (HCT116 or SW480). Mean fluorescence intensity (MFI) decrease in CD14 expression analyzed by flow cytometry. Concentrations of BM7PE were as indicated, 100 or 1000 ng/ml. The result is representative of triplicates. **** p<0.0001, ** p<0.0025.

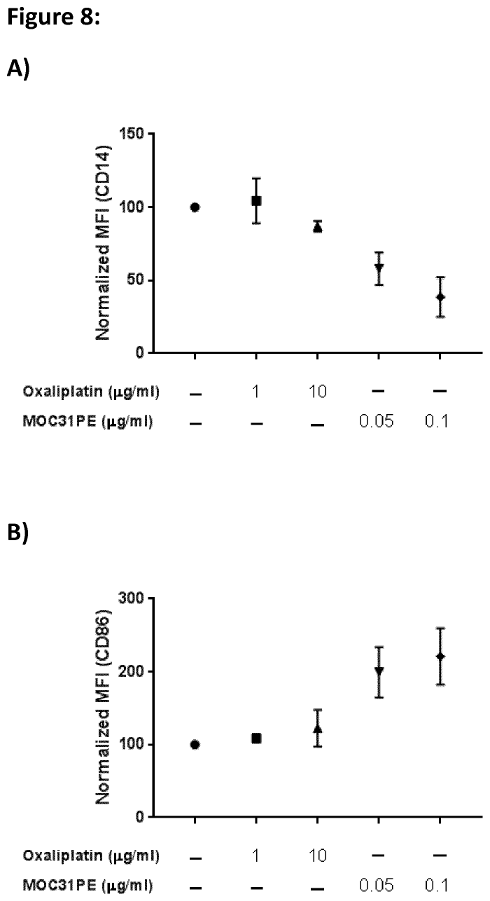

FIG. 8: Ex vivo maturation of immature dendritic cells (DCs) induced by MOC31PE.

Immature DCs treated with conditioned medium from MOC31PE treated colorectal cancer cells (HCT116) show a decrease in CD14 expression compared to immature DCs+conditioned medium from non-treated cells or cells treated with oxaliplatin (A) and show an increase in CD86 expression compared to immature DCs+conditioned medium from non-treated cells or cells treated with oxaliplatin (B). Mean fluorescence intensity (MFI) of CD14 expression and CD86 expression was analyzed by flow cytometry. Concentrations of MOC31PE were as indicated, 0.05 or 0.1 μg/ml. The data is from three biological triplicates, each in three technical triplicates.

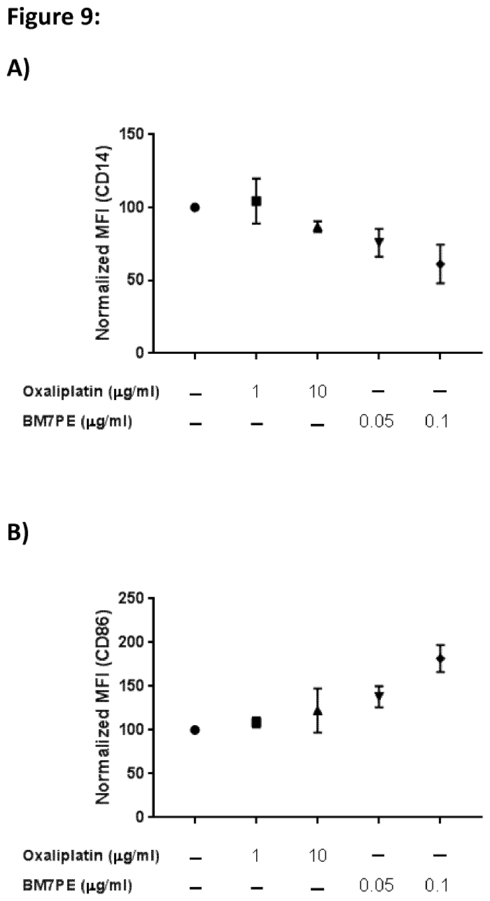

FIG. 9: Ex vivo maturation of immature dendritic cells (DCs) induced by BM7PE.

Immature DCs treated with conditioned medium from BM7PE treated colorectal cancer cells (HCT116) show a decrease in CD14 expression compared to immature DCs+conditioned medium from non-treated cells or cells treated with oxaliplatin (A) and show an increase in CD86 expression compared to immature DCs+conditioned medium from non-treated cells or cells treated with oxaliplatin (B). Mean fluorescence intensity (MFI) of CD14 expression and CD86 expression was analyzed by flow cytometry. Concentrations of BM7PE were as indicated, 0.05 or 0.1 μg/ml. The data is from three biological triplicates, each in three technical triplicates.

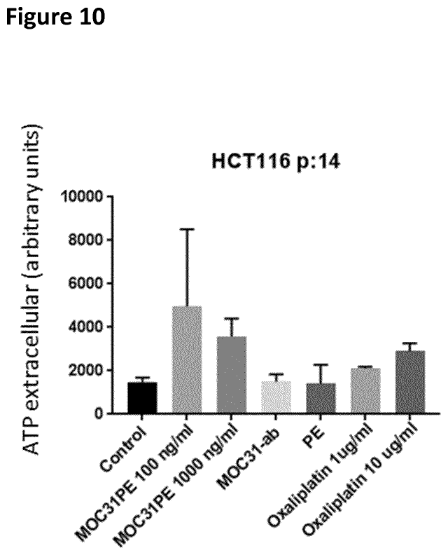

FIG. 10: MOC31PE-treated cancer cells release ATP ATP assay demonstrating that MOC31PE-treated HCT116 cells release ATP. For comparison, data is also shown for oxaliplatin, MOC31 antibody (MOC31-ab) and the PE toxin (PE).

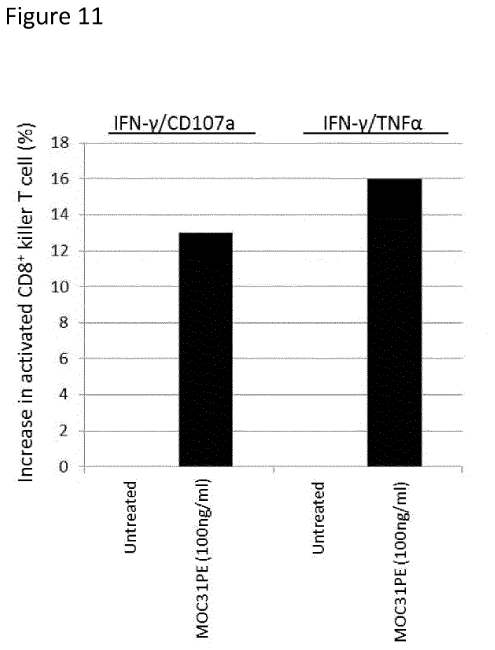

FIG. 11: MOC31PE promotes activation of killer T cells (CD8+) ex vivo

Bone marrow-derived monocytes were thawed and cultured with the cytokines IL-4+GM-CSF for 2 days to generate immature DCs. HCT116 cells were treated with immunotoxin (MOC31PE) for 24 hrs and then the isolated conditioning medium (i.e. the supernatant with the HCT116 cells removed) was co-incubated with immature DCs for 24 hrs. DCs were stained for CD14-FITC, CD86-PE and analysed by flow cytometry. A portion of the treated DCs were transferred to a T-cell culture. The T cells become activated by the mature DCs, and the activation was measured by expression of the degranulation marker CD107a (−Cy5) and IFNγ (−FITC), and TNFα (−PE) in a flow cytometry analysis.

### EXAMPLE 1

Abstract:

BACKGROUND: We recently performed a first-in-man phase I clinical trial of the anti-EpCAM immunotoxin MOC31 PE in patients with metastatic cancer. Patients were treated intravenously with MOC31 PE alone and in combination with the immunosuppressor Cyclosporin A (CsA), once every 2 weeks up to 4 times. MOC31PE was well tolerated, and only a rapidly reversible dose-limiting liver toxicity was noticed. No complete or partial response was observed, as assessed by CT 8 weeks after start of therapy. In the present study, however, we reveal an unexpected long survival time of MOC31 PE monotherapy treated patients.

METHODS: We performed a retrospective analysis of overall survival (OS) in the two study arms, MOC31 PE administered alone (34 patients) and in combination with CsA (23 patients). Hazard ratio (HR) was estimated from Cox models, survival curves by the Kaplan-Meier method, patient sera were analyzed by multiplex biometric ELISA-based immunoassay. The putative immunogenic effect of MOC31PE was analyzed in vitro by release of HMGB1, and ex vivo in a dendritic cell maturation assay.

FINDINGS: The median OS time for all patients treated with MOC31 PE alone was 12.7 months (95% CI=5.6-19.8 months) as compared to 6.2 months (95% CI=5.6-6.8 months) (p=0.066) for the MOC31 PE+CsA group. This finding was unexpected, as preclinical studies had indicated that CsA would improve efficacy and block generation of neutralizing anti-MOC31 PE antibodies. To avoid possible bias related to the heterogeneity of cancers in our study population, we analyzed specifically the results for patients with the most frequent tumor type; colorectal cancer (CRC). The Kaplan-Meier survival plot shows that CRC-patients treated with MOC31PE alone (n=15) had a median OS of 16.3 months (95% CI=5.6-27.0 months) compared to 6.0 months (CI=5.8-6.2 months) for CRC-patients in the combination group (n=18) (HR=0.248, 95% credible interval: 0.109-0.564). The cytokine Th1 profile obtained in patient sera indicates that MOC31PE induces a novel and previously unknown immunogenic cell death mechanism. This was further supported by data demonstrating that MOC31PE-induced cell death of colorectal cancer cells released immune stimulating factors that caused maturation of patient-derived immature dendritic cells ex vivo.

INTERPRETATION: The present results reveal an unpredicted clinical benefit of anti-EpCAM immunotoxin treatment, particularly in a group of patients with advanced cancer with very limited treatment alternatives.

### Introduction

One of the hallmarks of cancer is to escape anti-tumor immune response. Lately, various approaches to overcome immune resistance have been applied clinically in several tumor types. However, in spite of some cases with unforeseen durable responses only about 25% of all patients respond to immuno-oncology drugs. To improve response rates and overcome resistance new drugs and combinations are being evaluated in the clinic, and compounds with different mechanisms of immune-stimulation are investigated.

Recently, we reported the results of a phase I clinical trial with the EpCAM-targeting immunotoxin MOC31 PE, with and without parallel administration of the immunosuppressor Cyclosporin A (CsA) (1). The safety and maximum tolerated doses in patients with EpCAM-positive tumors of various types were determined, and only moderate and rapidly reversible dose-limiting liver toxicity was observed. Notably, the patients did not experience subjective side effects that could be ascribed to the treatment.

No complete or partial responses were obtained as determined by CT taken eight weeks after start of treatment. Nevertheless, in the current study we performed a detailed investigation of the data from the phase I trial and looked at the survival time of subgroups of patients. We found that the median overall survival time (OS) for all patients receiving the combination of MOC31PE and CsA was 6.2 months, as expected for this group of patients. By contrast, for the patients treated with MOC31 PE alone the median OS was more than 12 months. This difference was highly unexpected as in preclinical studies the combination was superior and even synergistic compared to the use of MOC31 PE alone (2). Based on this data, we hypothesized that MOC31PE might have induced an immunogenic response that was obliterated by the immunosuppression caused by CsA. When examining this possibility, we obtained results indicating that the MOC31PE immunotoxin represents a highly promising immunogenic stimulant, in addition to exerting its specific tumor cell killing capacity.

### Material and Methods

### Patients

The completed phase I trial comprised of two parts, the first with MOC31PE alone in 34 patients (modified Fibonacci dose escalation), and the second part in 23 patients with dose escalation of MOC31PE with concomitant administration of Sandimmune® (CsA) (1). In both treatment arms, MOC31PE was repeated every second week up to 4 times (day 1, 14, 28, 42). In the combination group, CsA was given one day before MOC31PE (day 0) and then for 4 subsequent days.

The inclusion criteria was EpCAM positive metastatic carcinoma, age 18 years or older, in patients with ECOG performance status 0-2. Before inclusion and study related investigations, the patients signed an approved written informed consent.

### MOC31PE

The MOC31 monoclonal mouse antibody (IgG1) recognizing the CD326 antigen (EpCAM) was produced and purified to clinical grade by MCA Development, Groningen, Netherlands. Pseudomonas exotoxin A (PE) (full-length, wild-type) was isolated from the fermentation broth of Pseudomonas aeruginosa PA103, manufactured at University of Ohio, Columbus, Ohio. The MOC31 PE conjugate was produced to clinical grade at Fred Hutchinson Cancer Research Center, Biologics Production Facility, Seattle, Wash. (1).

Further details in relation to the production of Pseudomonas exotoxin A (PE) and of how it is conjugated to the MOC31 antibody are given below.

Pseudomonas exotoxin A (PE) was isolated from the fermentation broth of P. aeruginosa PA 103 (6). PE is produced by inoculation of a New Brunswick in vitro fermentor with an overnight culture of P. aeruginosa for 18-20 hrs. at 32° C. at 400 rpm. The exotoxin A was purified from 50 L of culture supernatant according to the Leppla procedure (22) with a few modifications as described by Galloway et al. (23). All procedures were performed at 5° C. The cells were harvested, and the supernatant was diluted with cold deionized water (150 litres) to bring down the ionic concentration, and DEAE cellulose (DE-52; 2 litres) slurried in water, was added. While the suspension was vigorously stirred, HCl (2M; 150 ml) diluted in cold water (4 litres) was added. After 1 h of stirring, the DE-52 resin was allowed to settle for 2 h and then was collected and washed three times with cold Tris-hydrochloride (buffer A; 0.01 M; pH 8.1). Finally, the washed DE-52 resin was transferred to a funnel and slowly eluted with cold 0.15M Tris-hydrochloride (2.5 litres; pH 8.1). The first 500 ml of eluate was discarded. The remaining 2 litres was collected, and 2-mercaptoethanol was added to a final concentration of 5 mM. Exotoxin A was precipitated by the addition of solid ammonium sulphate to 75% saturation. The precipitate was redissolved and dialyzed overnight against buffer A containing 2-mercaptoethanol (2 mM). It was then applied to a DE-52 column (200 ml) equilibrated with the same buffer. The exotoxin A bound to the column, was eluted off the column using a linear gradient of 0.01 to 0.3 M NaCl in buffer A containing 2-mercaptoethanol (2 mM). The exotoxin A fractions that were pooled were adjusted to pH 6.8 with 2M HCl.

Exotoxin A was further purified by applying to a column of hydroxylapatite equilibrated with sodium phosphate buffer (5 mM)/sodium chloride (50 mM) at pH 7.0. The bound exotoxin A was eluted off the column by a linear gradient of sodium phosphate, 5 mM to 100 mM in 50 mM sodium chloride at pH 7.0. The protein peak emerging at 40-60 mM sodium phosphate was concentrated by precipitation with ammonium sulphate, redissolved and dialyzed against buffer A containing 2-mercaptoethanol (2 mM). The exotoxin A thus purified was aliquoted and stored frozen at −70° C.

The MOC31 antibody was conjugated to the PE by a thioether bond. More specifically, MOC31 antibody that had been treated (or derivatised) using DTT (dithiothreitol) was conjugated to PE that had been derivatised using the reagent SMCC (succinimidyl-4-(N-maleimidomethyl) cyclohexane-1-carboxylate).

### Ethical

The clinical phase I study was approved by the Norwegian Medicines Agency, by the Norwegian Regional Ethical Committee (REC) and by the institutional review board. The registration number is NCT01061645 with the study title “Study of MOC31-PE in antigen positive carcinomas”. All patients provided written informed consent.

### Cytokine Measurement

Serum samples were taken prior to dosing and stored frozen at −70° C. All samples were thawed on ice, vortexed, spun down at 14,000×g for 10 min at 4° C. and tested in a multiplex biometric ELISA-based immunoassay. The cytokines included: interleukin-1 β (IL-1β), interleukin-2 (IL-2), interleukin-6 (IL-6), interleukin-12 (IL-12), granulocyte-macrophage colony-stimulating factor (GM-CSF), tumour necrosis factor α (TNF-α), and interferon-γ (IFN-γ), according to the manufacturer's instructions (Bioplex, Bio-Rad Lab., Inc., Hercules, Calif., USA). Serum levels of all proteins were determined using the Bio-Plex array reader Luminex IS 100 instrument (Luminex, Austin, Tex., USA) that quantifies multiplex immunoassays in a 96-well plate. The analyte concentration was calculated using a standard curve, with software provided by the manufacturer.

### Cell Lines

Colon cancer cell lines HCT116, SW480 and HT29 (ATCC, Rockville, Md.) were cultured in RPMI-1640 medium supplemented with Hepes, Glutamax (all from Lonza, Austria), 10% heat-inactivated FCS (PAA, GE Healthcare, UK) at 37° C. All cell lines were routinely tested and found to be free from contamination with Mycoplasma species. The cells were routinely ID tested.

### Cell Viability

The Cell Titer 96 AqueousOne solution assay (MTS (Promega Madison, Wis.)) was used to determine cell viability as previously described (3). Cells (10 000) were seeded in 96-well plates. After 24 h incubation, the medium was replaced with medium containing MOC31PE (1-1000 ng/ml), and incubated further for 24 h. The MTS solution was then added to the wells, and the absorbance was measured 2 h later at a wavelength of 490 nm. The viability of MOC31PE treated cells were compared to the values for untreated control cells and recorded as the percentage cell viability of control cells. The assays were performed in triplicate.

### Western Blotting for HMGB1 Detection

HCT116 cells (2×10⁶) were seeded in T-25 flasks and after 24 h, treated with MOC31PE (10 and 100 ng/ml) or mAb MOC31 (100 ng/ml) or vehicle (PBS, 0.1% HSA) for 24 h. Conditioned media from cells were centrifuged and supernatants were collected. Equal volumes of supernatant were separated by NuPAGE Bis-Tris gel (Invitrogen, Carlsbad, Calif.), and subsequently transferred by electrophoresis to Immobilon membrane (Millipore, Bedford, Mass.). The membrane was blocked with 5% nonfat dry milk for 1 h at room temperature followed by incubation with rabbit anti-HMGB1 (Cell Signaling Technology (Danvers, Mass.)) in 5% w/v BSA, 1×TBS, 0.1% Tween-20 at 4° C. overnight. The membranes were washed before incubation with appropriate HRP-coupled secondary antibodies. Following several washes, the peroxidase activity was visualized with enzyme-linked chemiluminescence (ECL, Amersham Pharmacia Biotech, Buckinghamshire, UK.)

### Generation of Dendritic Cells

Immature dendritic cells (DCs) were generated essentially as described in Subklewe, et al. (4). Briefly, monocytes obtained from leukapheresis product (REC Project no: 2013/624-15) were cultured for 2 days with GM-CSF and Interleukin-4 (IL-4) in Ultra-low attachment cell culture flasks (Corning). Cancer cell lines were treated for 24 h or 48 h with MOC31PE, 9.2.27PE, and MOC31 at indicated concentrations.

The immature DCs were then either matured for 24 h with MOC31PE-treated colon cancer cell lines or their supernatants (sn) in 96-well plates. As a positive control, cytokines facilitating maturation were used (IL-113, IL-6, TNF-α, IFN-γ (all from PeproTech, Rocky Hill, N.J.), prostaglandin E₂(PGE2), and TLR7/8 agonist R848 (MedChem Express, Sweden) (5). Immature DCs cultured with IL-4 and GM-CSF were used as negative control. The mature DC phenotype was evaluated by flow cytometry.

### Flow Cytometry

Cells were washed in staining buffer consisting of phosphate buffered saline (PBS) containing 2% FCS before staining with CD9-BV510 (M-L13, BD Biosciences, San Jose, Calif.) and CD14-FITC (61D3, Thermo Fisher Scientific Inc, Waltham, Mass.). Finally, cells were resuspended in staining buffer containing 1% paraformaldehyde. Samples were acquired on a LSR II flow cytometer (BD Bioscience) and the data were analyzed using FlowJo software (Treestar Inc., Ashland, Oreg.). An analogous experiment was also performed using a CD86 antibody to analyse CD86 expression.

### Statistical Analysis

Survival was estimated by the Kaplan-Meier method and survival curves compared using the Log-rank test. Univariate analysis was conducted by Cox proportional hazards regression.

The non-parametric two-tailed Mann-Whitney test was used to compare the median values of cytokine variables in the pre- and post-treatment groups. All statistical analyses were performed using the SPSS statistical package (version 21, SPSS, Chicago, Ill.). The level of statistical significance for the cytokine analysis was set at p<0.05.

All statistical analyses for DC maturation were performed using GraphPad Prism® (GraphPad Software, Inc.). Unpaired t-tests were used for comparison of DC maturation between conditions and all p-values given are two-tailed values. The level of statistical significance for the DC maturation analysis was set to p<0.05.

### Results

### Overall Survival

In our recent phase I trial, the toxicity of systemic therapy with MOC31 PE alone and in combination with cyclosporin A was modest and the maximum tolerated dose (MTD) was determined (1). No objective tumor responses were found as judged by CT scans eight weeks after the first MOC31 PE infusion (1).

We reevaluated our data at a later date and noticed that the median OS time for all patients treated with MOC31PE alone was 12.7 months (95% CI=5.6-19.8 months) as compared to 6.2 months (95% CI=5.6-6.8 months) (p=0.066) for the MOC31PE+CsA group (FIG. 1a). This difference was unexpected, as in preclinical studies the combination was superior, in fact synergistic to MOC31 PE alone both in vitro and in human tumor xenograft models (2). Therefore, we hypothesized that the observed OS difference could indicate that MOC31PE had induced an immunogenic anti-tumor effect that was obliterated by the immunosuppression caused by concomitant CsA. To examine this possibility, and to avoid bias related to the heterogeneity of tumor types in our study population, we analyzed specifically the results for patients with the most frequent tumor type; colorectal cancer. Of these, 15 patients had been treated with monotherapy and 18 with the combination. The Kaplan-Meier survival plot (FIG. 1b) shows that patients treated with MOC31PE alone had a median OS of 16.3 months (95% CI=5.6-27.0 months) compared to 6.0 months (CI=5.8-6.2 months) for patients in the MOC31 PE plus CsA group, which is comparable to OS of untreated patients with progressive disease on last line of standard chemotherapy (7, 8). In a univariable analysis, hazard ratio (HR) with 95% credible interval (Crl) was calculated for monotherapy vs combination (HR=0.248, 95% Crl: 0.109-0.564). In addition, when we excluded the five patients in the MOC31 PE monotherapy group who had received additional treatment after MOC31 PE (two with only local radiotherapy for bone metastasis) the median OS remained longer than expected (12 months, data not shown).

### MOC31 PE Induced Release of HMGB1 In Vitro

To further evaluate the putative immunogenic effect of MOC31PE we wanted first to demonstrate its ability to kill colon cancer cell lines as a background for studying possible immunogenic cell death (ICD) factors such as the non-histone chromatin-binding nuclear protein high-mobility group box 1 (HMGB1) (9). In a dose-dependent manner, MOC31PE effectively decreased the cell viability of the colorectal cancer cell lines HCT116, SW480 and HT29 (FIG. 3a), with ID₅₀ values similar for all three cell lines (100 ng/ml). Of note, release of HMGB1 was found in the supernatant of the MOC31PE treated HCT116 cells (FIG. 3b), as detected by western blot, but not in the supernatant of cells treated with the naked antibody MOC31 (100 ng/ml). Release of HMGB1 also was found in the supernatant of the MOC31PE treated SW480 cells (FIG. 3b), as detected by western blot.

It was also found that HMGB1 was released from MOC31PE treated MA-11 cells (a metastatic breast cancer cell line) (data not shown).

### MOC31PE-Treated Cancer Cells Secrete Factors Responsible for Ex Vivo Maturation of Dendritic Cells

The CD14 protein level, a well known marker of immature DCs, decreases during DC maturation. In the ex vivo system used here, the CD14 level was significantly reduced by the addition of conditioned medium from MOC31PE-treated colon cancer cells (HCT116 and SW480) (p<0.0025) (FIG. 4) to an extent similar to that of the positive control (FIG. 4). The effect was MOC31PE dose-dependent, with increasing maturation of DCs with increasing MOC31PE concentrations. The irrelevant immunotoxin 9.2.27PE(10) (recognizing antigen HMW-MAA a melanoma specific antigen not expressed by colorectal cancer cells) as well as the MOC31 antibody did not kill colon cancer cells, and the supernatant from 9.2.27PE and MOC31 treated cells had no maturation effect on immature DCs. The results demonstrate that MOC31PE induces release of immune stimulating factors causing maturation of immature DCs.

Further experiments were performed which further demonstrate that MOC31PE-treated colon cancer cells release factors (immunogenic cell death factors) responsible for the ex vivo maturation of dendritic cells. The results show that conditioned media from MOC31PE-treated HCT-116 cells increases the maturation of dendritic cells (DCs) (FIG. 8A and FIG. 8B). Immature (imm) DCs were isolated from human monocytes from leukapheresis products by elutriation, and differentiated with GM-CSF and IL-4 into immDCs. The CD14 protein level, a well-known marker of immature DCs, decreases during DC maturation. The CD86 protein level, a well-known marker of mature DCs, increases during DC maturation. Interestingly, the very well-known ICD-inducer oxaliplatin, did not activate (i.e. did not enhance the maturation of) the immDCs as well as MOC31 PE (FIG. 8A and FIG. 8B). The effect was MOC31 PE dose-dependent, with increasing maturation of DCs with increasing MOC31 PE concentrations (FIG. 8A and FIG. 8B). The irrelevant immunotoxin 9.2.27PE (recognizing antigen HMW-MAA a melanoma specific antigen not expressed by colorectal cancer cells) as well as the MOC31 antibody did not kill colon cancer cells, and the supernatant from 9.2.27PE and MOC31 treated cells had no maturation effect on immature DCs (data not shown). The results demonstrate that MOC31 PE induces release of immune stimulating factors causing maturation of immDCs. The data in FIGS. 8A and 8B is from three biological triplicates, each in three technical triplicates.

### Cytokines in Patients' Serum

The levels of pro-inflammatory Th1-type cytokines IL-2, IL-6, IL-12, IFN-γ and TNF-α, and the Th1 related IL-1β and GM-CSF were examined in serum from the patients with colorectal cancer: pretreatment (n=20), post-MOC31PE and post-MOC31PE+cyclosporin (in both cases 2 weeks after first immunotoxin administration, n=12).

With the exception of IL-6, the levels of all cytokines increased significantly in serum from MOC31 PE treated patients relative to the corresponding pre-treatment levels (p<0.01) (FIG. 2). Also, IL-2 (p<0.05), IL-12 (p<0.01) and GM-CSF (p<0.01) levels were significantly higher in the serum from MOC31PE treated patients compared to the combination group (FIG. 2). In table 1, the median and minimum and maximum ranges in cytokine concentration are presented. A large fraction of the pre-treatment samples had cytokine levels below the detection sensitivity of the assay. One patient in the monotherapy group (11%) and three in the combination group (25%) had all cytokine levels close to zero, the reason for which is unknown.

The results demonstrate that the MOC31 PE did induce a significant Th1 cytokine response in most patients which was abolished by the addition of CsA.

TABLE 1
Serum cytokines levels.

| | Cytokine(pg/ml) | | | | | | | | | | | | | |
|---|---|---|---|---|---|---|---|---|---|---|---|---|---|---|
| | IL1b (pg/ml) | | IL2 (pg/ml) | | IL6 (pg/ml) | | IL12 (pg/ml) | | GMCSF(pg/ml) | | IFN γ (pg/ml) | | TNFα (pg/ml) | |
| | Median | Range | Median | Range | Median | Range | Median | Range | Median | Range | Median | Range | Median | Range |
| Pre | 0 | 0-23 | 0 | 0-322 | 4 | 0-109 | 4 | 0-278 | 4 | 0-166 | 0 | 0-393 | 0 | 0-153 |
| (n = 20) | | | | | | | | | | | | | | |
| Post | 12 | 0-576 | 58 | 0-824 | 29 | 0-1508 | 118 | 3-2603 | 165 | 7-1495 | 394 | 9-7912 | 158 | 0-6893 |
| MOC31PE | | | | | | | | | | | | | | |
| (n = 9) | | | | | | | | | | | | | | |
| Post | 3 | 0-31 | 10 | 0-75 | 16 | 0-67 | 7 | 1-173 | 34 | 2-167 | 111 | 0-1756 | 17 | 0-242 |
| MOC31PE + | | | | | | | | | | | | | | |
| CsA | | | | | | | | | | | | | | |
| (n = 12) | | | | | | | | | | | | | | |

Serum from patients, taken before (n = 20) and two weeks after MOC31PE (n = 9) or MOC31PE plus CsA (n = 12) administration, analyzed by multiplex cytokine ELISA assay. The data is presented as the median and range of the individual cytokine levels (pg/ml).

### Discussion

The clinical use of immunotoxins in cancer treatment has been hampered by their high immunogenicity leading to generation of anti-immunotoxin antibodies (11). We have demonstrated in preclinical experiments that combination therapy with CsA could circumvent this problem by inhibiting/delaying anti-immunotoxin antibody production, and this was confirmed in our phase I study (1, 2). However, in contrast to our expectation that the combination also could lead to increased anti-tumor efficacy, similar to that observed in preclinical experiments, we found the opposite, an observed long OS in patients treated with the MOC31 PE alone, compared to OS of those also receiving CsA. The present study demonstrates the novel and exciting finding that MOC31PE induces a strong immunogenic effect and prolonged overall survival of patients, e.g. patients with advanced colorectal cancer, a group of patients with very limited treatment alternatives.

In addition to the i.v. administered MOC31 PE clinical phase I trial, we have recently completed a clinical phase I study with intraperitoneal MOC31PE treatment of patients with colorectal cancer peritoneal metastases undergoing cytoreductive surgery and hyperthermic intraperitoneal chemotherapy (12). Also in this study, the immunotoxin treatment was well tolerated, supporting the safety of MOC31 PE treatment.

Previous clinical experience with immunotoxins, commonly highly modified or recombinant, has been disappointing (11). The underlying reasons for this might include pharmacokinetic limitations with short half-lives and low levels of active plasma concentrations, resulting in frequent injections and often severe toxicity. We report here that an immunotoxin, that has a fully murine antibody and non-modified PE conjugated with a stable and strong thioether bond, exerts an immunogenic effect.

The large size of the molecule is thought to prevent penetration of MOC31 PE deep into large solid tumor lesions. However, the immunogenic effect, and the long OS, seems to depend on specific binding of the immunotoxin to tumor cells expressing its target antigen, followed by killing of accessible tumor cells. Without wishing to be bound by theory, our data suggests that MOC31PE is inducing immunogenic cell death, involving stimulating maturation of dendritic cells and activation of T cells. The presence and increase in the levels of a number of cytokines in the serum of MOC31PE only treated patients support this. We found increased levels of IL-12 and IFNγ, both inducers of Th1 cells and necessary to promote potent cytotoxic T cells that are accountable for antitumor immunity (13). IL-2, another cytokine induced by MOC31PE, has been shown to be crucial to the expansion of CD8⁺ T cells and is particularly important for the functional maturation of activated T cells. In support of induction of this cytokine response being caused by MOC31PE-initiated ICD, we detected the release of HMGB1, one of the hallmarks of ICD (9), from colon cancer cells treated with MOC31 PE.

The immense success of checkpoint inhibitors in the clinic has been limited by aberrant immune checkpoint inhibition and/or absence of appropriate co-stimulation. This is observed in several cancer types with only a subset of patients responding and experiencing long term disease-free or OS. Several explanations have been suggested, including the need for stimulating the CD28 receptor (14), epigenetic stability of exhausted T-cells (15, 16), and the effect of the chaperone protein HSP90 altering the effect of proteins produced by mutated tumor genes (17). The limitations of individual checkpoint inhibitors have prompted the need for combination therapies, both with other inhibitors and with chemo- and radio-therapy (18-20).

MOC31PE has a mechanism of action that is different from checkpoint inhibitors; inhibiting protein synthesis and inducing apoptosis (2, 21). This is in addition to the immunogenic cell death anti-cancer effect described in the present study. The immunotoxin has furthermore an attractive safety profile with no subjective side effects and only modest liver toxicity. This makes it an attractive candidate for combination with checkpoint inhibitors. In summary, the MOC31PE immunotoxin represents a novel and highly promising immunostimulant for use in cancer therapies, particularly in patients with colorectal cancer.

### REFERENCES

1. Y. Andersson et al., Phase I trial of EpCAM-targeting immunotoxin MOC31 PE, alone and in combination with cyclosporin. British journal of cancer 113, 1548-1555 (2015).
2. Y. Andersson, O. Engebraaten, O. Fodstad, Synergistic anti-cancer effects of immunotoxin and cyclosporin in vitro and in vivo. British journal of cancer 101, 1307-1315 (2009).
3. Y. Andersson, H. Le, S. Juell, O. Fodstad, AMP-activated protein kinase protects against anti-epidermal growth factor receptor-Pseudomonas exotoxin A immunotoxin-induced MA11 breast cancer cell death. Molecular cancer therapeutics 5, 1050-1059 (2006).
4. M. Subklewe et al., Dendritic cell maturation stage determines susceptibility to the proteasome inhibitor bortezomib. Human immunology 68, 147-155 (2007).
5. S. Spranger et al., Generation of Th1-polarizing dendritic cells using the TLR7/8 agonist CL075. Journal of immunology 185, 738-747 (2010).
6. Leppla, S. H.: Large scale purification and characterization of the exotoxin A of Pseudomonas aeruginosa. Infect & Immunity. 14: 1077-1086.
7. A. Grothey et al., Regorafenib monotherapy for previously treated metastatic colorectal cancer (CORRECT): an international, multicentre, randomised, placebo-controlled, phase 3 trial. Lancet (London, England) 381, 303-312 (2013).
8. R. J. Mayer et al., Randomized trial of TAS-102 for refractory metastatic colorectal cancer. The New England journal of medicine 372, 1909-1919 (2015).
9. O. Kepp et al., Consensus guidelines for the detection of immunogenic cell death. Oncoimmunology 3, e955691 (2014).
10. K. Risberg, O. Fodstad, Y. Andersson, The melanoma specific 9.2.27PE immunotoxin efficiently kills melanoma cells in vitro. International journal of cancer 125, 23-33 (2009).
11. I. Pastan, R. Hassan, D. J. Fitzgerald, R. J. Kreitman, Immunotoxin therapy of cancer. Nature reviews. Cancer 6, 559-565 (2006).
12. A. Y. Frøysnes I S, Larsen S G, Davidson B, └ien J M T, Olsen K H, Giercksky K E, Julsrud L, Fodstad Ø, Dueland S, Flatmark K., Novel treatment with intraperitoneal MOC31PE immunotoxin in colorectal peritoneal metastasis—results from the ImmunoPeCa phase I trial Annals of Surgical Oncology (2017).
13. A. D. Garg, D. De Ruysscher, P. Agostinis, Immunological metagene signatures derived from immunogenic cancer cell death associate with improved survival of patients with lung, breast or ovarian malignancies: A large-scale meta-analysis. Oncoimmunology 5, e1069938 (2016).
14. M. Janakiram et al., The third group of the B7-CD28 immune checkpoint family: HHLA2, TMIGD2, B7x, and B7-H3. Immunological reviews 276, 26-39 (2017).
15. K. E. Pauken et al., Epigenetic stability of exhausted T cells limits durability of reinvigoration by PD-1 blockade. Science (New York, N.Y.) 354, 1160-1165 (2016).
16. R. A. Amezquita, S. M. Kaech, Immunology: The chronicles of T-cell exhaustion. Nature 543, 190-191 (2017).
17. M. L. Siegal, Molecular genetics: Chaperone protein gets personal. Nature 545, 36-37 (2017).
18. J. Lohmueller, O. J. Finn, Current modalities in cancer immunotherapy: Immunomodulatory antibodies, CARs and vaccines. Pharmacology & therapeutics, (2017).
19. T. Kuusk et al., Antiangiogenic therapy combined with immune checkpoint blockade in renal cancer. Angiogenesis, (2017).
20. A. Esposito, C. Criscitiello, G. Curigliano, Immune checkpoint inhibitors with radiotherapy and locoregional treatment: synergism and potential clinical implications. Current opinion in oncology 27, 445-451 (2015).
21. Y. Andersson, S. Juell, O. Fodstad, Downregulation of the antiapoptotic MCL-1 protein and apoptosis in MA-11 breast cancer cells induced by an anti-epidermal growth factor receptor-Pseudomonas exotoxin a immunotoxin. International journal of cancer 112, 475-483 (2004).
22. Galloway, D. R., Hedstrom, R. C., McGowan, J. L., Kessler, S. P. and Wozniak, D. J.: Biochemical analysis of CRM66: a nonfunctional Pseudomonas aeruginosa exotoxin A. J. Biol. Chem. 264: 14869-14873.
23. Morgan, A. C., Sivam, G. P., Beaumier, P., McIntyre, R. Abrams, P. G.: Immunotoxins of Pseudomonas exotoxin A (PE): Effect of linkage on conjugate yield, potency, selectivity and toxicity. Molecular Immunology. 27: 273-282, 1990.

### EXAMPLE 2

Experiments were also performed with the immunotoxin BM7PE, looking at its effect on protein synthesis, cell viability and the maturation of dendritic cells.

### Material and Methods

### BM7PE

BM7PE is an immunotoxin in which the antibody component is the BM7 antibody and the toxic biological molecule is PE (full-length PE). The PE was produced as described in Example 1 and was conjugated to the BM7 antibody in an analogous manner to that described in Example 1 in relation to MOC31 PE.

### Cell Viability

The Cell Titer 96 AqueousOne solution assay (MTS (Promega Madison, Wis.)) was used to determine cell viability as previously described. Cells (10 000) were seeded in 96-well plates. After 24 h incubation, the medium was replaced with medium containing BM7PE (0.01-1000 ng/ml), and incubated further for 24 h (T47D) or 48 h (HT116, SW480 and HT29). The MTS solution was then added to the wells, and the absorbance was measured 2 h later at a wavelength of 490 nm. The viability of BM7PE treated cells were compared to the values for untreated control cells and recorded as the percentage cell viability of control cells. The assays were performed in triplicate.

### Measurement of Protein Synthesis Inhibition

Protein synthesis inhibition caused by BM7PE was measured by using the [3H]-leucine incorporation assay (Sandvig & Olsnes, 1982, J. Biol. Chem., 257. p 7495-7503). Cells (4×10⁴ per well) were seeded in 48-well plates and allowed to grow overnight before addition of different concentrations of BM7PE. After 20 hr incubation, the cells were washed twice with cold phosphate-buffered saline (PBS), 0.1% FCS and incubated with [3H]leucine (2 μCi/ml) in leucine-free medium for 45 min at 37° C. The cells were then washed with 5% trichloroacetic acid (TCA) for 10 and 5 min, respectively, and dissolved in 0.1 M KOH for at least 5 min. The resultant solution was transferred to the liquid scintillator Aquasafe 300 Plus (Zinsser Analytic, Frankfurt/Main, Germany). Sample counts were determined in a liquid scintillation counter (LKB Wallac). Assays were performed in duplicate, and repeated at least three times.

### Generation of Dendritic Cells

Immature dendritic cells (DCs) were generated essentially as described in Subklewe, et al. (supra). Briefly, monocytes obtained from leukapheresis product (REC Project no: 2013/624-15) were cultured for 2 days with GM-CSF and Interleukin-4 (IL-4) in Ultra-low attachment cell culture flasks (Corning). Cancer cell lines were treated for 48 h with BM7PE at indicated concentrations.

The immature DCs were then either matured for 24 h with BM7PE-treated colon cancer cell lines supernatants (sn) in 96-well plates. As a positive control, cytokines facilitating maturation were used (IL-1β, IL-6, TNF-α, IFN-γ (all from PeproTech, Rocky Hill, N.J.), prostaglandin E₂(PGE2), and TLR7/8 agonist R848 (MedChem Express, Sweden)). Immature DCs cultured with IL-4 and GM-CSF were used as negative control. The mature DC phenotype was evaluated by flow cytometry.

### Flow Cytometry

Cells were washed in staining buffer consisting of phosphate buffered saline (PBS) containing 2% FCS before staining with CD9-BV510 (M-L13, BD Biosciences, San Jose, Calif.) and CD14-FITC (6ID3, Thermo Fisher Scientific Inc, Waltham, Mass.). Finally, cells were resuspended in staining buffer containing 1% paraformaldehyde. Samples were acquired on a LSR II flow cytometer (BD Bioscience) and the data were analyzed using FlowJo software (Treestar Inc., Ashland, Oreg.). An analogous experiment was also performed using a CD86 antibody to analyse CD86 expression.

### Results

### In Vitro Inhibition of Protein Synthesis and Cell Viability

In a dose-dependent manner, BM7PE effectively decreased protein synthesis (FIG. 5A) and the cell viability (FIG. 5B) of the breast cancer cell line T47D. For the colorectal cancer cell lines HCT116, SW480 and HT29 the BM7PE effectively decreased cell viability in all three cell lines (FIG. 6).

### BM7PE-Treated Cancer Cells Secrete Factors Responsible for Ex Vivo Maturation of Dendritic Cells

The CD14 protein level, a well known marker of immature DCs, decreases during DCs maturation. In the ex vivo system used here, the CD14 level was significantly reduced by the addition of conditioned medium from BM7PE-treated colon cancer cells (HCT116 and SW480) (p<0.0025) (FIG. 7) to an extent similar to that of the positive control (FIG. 7). The effect was BM7PE dose-dependent, with increasing maturation of DCs with increasing BM7PE concentrations. The irrelevant immunotoxin 9.2.27PE (recognizing antigen HMW-MAA a melanoma specific antigen not expressed by colorectal cancer cells) did not kill colon cancer cells, and the supernatant from 9.2.27PE treated cells had no maturation effect on immature DCs. The results demonstrate that BM7PE induces release of immune stimulating factors causing maturation of immature DCs.

Further experiments were performed which further demonstrate that BM7PE-treated colon cancer cells release factors (immunogenic cell death factors) responsible for the ex vivo maturation of dendritic cells. The results show that conditioned media from BM7PE-treated HCT-116 cells increases the maturation of dendritic cells (DCs) (FIG. 9A and FIG. 9B). Immature (imm) DCs were isolated from human monocytes from leukapheresis products by elutriation, and differentiated with GM-CSF and IL-4 into immDCs. The CD14 protein level, a well-known marker of immature DCs, decreases during DC maturation. The CD86 protein level, a well-known marker of mature DCs, increases during DC maturation. Interestingly, the very well-known ICD-inducer oxaliplatin, did not activate (i.e. did not enhance the maturation of) the immDCs as well as BM7PE (FIG. 9A and FIG. 9B). The effect was BM7PE dose-dependent, with increasing maturation of DCs with increasing BM7PE concentrations (FIG. 9A and FIG. 9B). The irrelevant immunotoxin 9.2.27PE (recognizing antigen HMW-MAA a melanoma specific antigen not expressed by colorectal cancer cells) as well as the BM7 antibody did not kill colon cancer cells, and the supernatant from 9.2.27PE and BM7 treated cells had no maturation effect on immature DCs (data not shown). The results demonstrate that BM7PE induces release of immune stimulating factors causing maturation of immDCs. The data in FIGS. 9A and 9B is from three biological triplicates, each in three technical triplicates.

### EXAMPLE 3

### MOC3IPE Induced Release of ATP In Vitro

### Determination of the Immunogenic Cell Death Marker—ATP.

Colon cancer cell lines HCT116 and SW480 were cultured according to manufacture (ATCC, Rockville, Md.). HCT116 cells were treated for 24 h with MOC31 PE and conditioned media from cells treated with MOC31PE (100 and 1000 ng/ml), mAb MOC31 (100 ng/ml) or vehicle (PBS, 0.1% HSA) were centrifuged. Equal volumes of cell supernatant were analyzed for the levels of secreted ATP in the supernatant by using the CellTiter-Glo® Luminescent Cell Viability Assay (Promega Madison).

The inventors have shown that the Damage Associated Molecular Patterns (DAMPs) HMGB1 and ATP, which are immunogenic cell death signals, are released by MOC31-PE-treated cancer cells (HCT116 cells and SW480 cells). FIG. 3(b) of Example 1 demonstrates the release of HMGB1. FIG. 10 shows, using an ATP assay, that MOC31PE-treated cancer cells release ATP. FIG. 10 also includes data for the ICD inducer oxaliplatin for comparison. The data in FIG. 10 indicates that MOC31 PE acts an immunogenic cell death ICD inducer. The monoclonal MOC31 antibody (MOC3-ab) and the toxin PE did not themselves act as ICD inducers.

### EXAMPLE 4

### MOC31PE Promotes Activation of Killer T Cells (CD8+) Ex Vivo

### Materials and Methods

Bone marrow-derived monocytes were thawed and cultured with the cytokines IL-4+GM-CSF for 2 days to generate immature DCs. HCT116 cells were treated with immunotoxin (MOC31PE) for 24 hrs and then the isolated conditioning medium (i.e. the supernatant with the HCT116 cells removed) was co-incubated with immature DCs for 24 hrs. DCs were stained for CD14-FITC, CD86-PE and analysed by flow cytometry. A portion of the treated DCs were transferred to a T-cell culture. The T cells becomes activated by the mature DCs, and the activation was measured by expression of the degranulation marker CD107a (−Cy5) and IFNγ (−FITC), and TNFα (−PE) in a flow cytometry analysis (CD107a (−Cy5) and IFNγ (−FITC), and TNFα (−PE) were run in the same flow as a triple stain).

### Results

ICD-factors released into the conditioning medium by MOC31PE-treated HCT116 cancer cells are able to activate immature DC to mature DC (see Example 1), which will activate T-cells leading to an increase in the population of the killer T cells (CD8+) ex vivo. This is illustrated by the data in FIG. 11. The CD8+ T-cell population (expressing the markers IFNγ, CD107a and TNFα) was analyzed by flow cytometry. The conditioning medium from untreated HCT116 cancer cells had no effect on DC and no effect on T-cells (as shown in FIG. 11). Without wishing to be bound by theory, it is believed that, in vivo, treatment with the MOC31 PE immunotoxin would result in an increase in the killer T cell (CD8+) population size and these T-cells will seek out and destroy tumor cells (e.g. metastatic tumour cells) and reduce tumor lesions, even solid tumors, by infiltrating it and releasing killing factors.

1-29. (canceled)

30. A method of treating a carcinoma in humans, said method comprising systemically administering to a human in need thereof a therapeutically effective amount of an immunotoxin that induces immunogenic cell death of cancer cells, wherein the immunotoxin comprises a murine antibody conjugated to a full-length Pseudomonas exotoxin A (PE), wherein said murine antibody binds to EpCAM or to MUC-1 and comprises an Fc domain, and wherein said immunotoxin
(i) is used in combination with an immunostimulant; or
(ii) is the sole active agent used in the treatment regimen.

31. The method of claim 30, wherein said carcinoma is colon carcinoma.

32. The method of claim 30, wherein said carcinoma is a metastatic and/or advanced carcinoma.

33. The method of claim 30, wherein said carcinoma is an EpCAM-positive carcinoma.

34. The method of claim 30, wherein said carcinoma is a MUC-1-positive carcinoma.

35. The method of claim 30, wherein said murine antibody is a full-length antibody, preferably an IgG antibody, more preferably an IgG₁ antibody.

36. The method of claim 30, wherein said full-length Pseudomonas exotoxin A (PE) has the amino acid sequence set forth in SEQ ID NO: 3.

37. The method of claim 30, wherein said murine antibody that binds to EpCAM is the antibody MOC31.

38. The method of claim 30, wherein said immunotoxin is MOC31PE.

39. The method of claim 30, wherein said murine antibody that binds to MUC-1 is the antibody BM7.

40. The method of claim 30, wherein said immunotoxin is BM7PE.

41. The method of claim 30, wherein said immunostimulant is a checkpoint inhibitor.

42. The method of claim 41, wherein said checkpoint inhibitor is a PD-1 antibody, a PD-L1 antibody or a CTLA-4 antibody.

43. The method of claim 41, wherein said checkpoint inhibitor is selected from the group consisting of Nivolumab, Pembrolizumab, Ipilimumab, Atezolizumab, Durvalumab, Avelumab and Cemiplimab.

44. The method of claim 30, wherein said immunotoxin is administered intravenously.

45. A method for stimulating an immune response in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, and wherein said immunotoxin
(i) is used in combination with an immunostimulant; or
(ii) is the sole active agent used in the treatment regimen.

46. A method for inducing immunogenic cell death in a human subject, said method comprising administering an immunotoxin to said subject, wherein the immunotoxin comprises a murine antibody conjugated to a full-length Pseudomonas exotoxin A (PE), wherein said murine antibody binds to EpCAM or to MUC-1 and comprises an Fc domain, wherein said immunotoxin is systemically administered, and wherein said immunotoxin
(i) is used in combination with an immunostimulant; or
(ii) is the sole active agent used in the treatment regimen.


SEQUENCE LISTING (3 sequences)

SEQ ID NO: 1 - protein, 116 aa, mouse
QVKLQQSGPELKKPGETVKISCKASGYTFTNYGMNWVKQAPGKGLKWMGWINTYTGESTYADDFKGRFAFSLETSASAAYLQINNLKNEDTATYFCARFAIKGDYWGQGTTVTVSS

SEQ ID NO: 2 - protein, 113 aa, mouse
DIVLTQSPFSNPVTLGTSASISCRSTKSLLHSNGITYLYWYLQKPGQSPQLLIYQMSNLASGVPDRFSSSGSGTDFTLRISRVEAEDVGVYYCAQNLEIPRTFGGGTKLEIKR

SEQ ID NO: 3 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK